(19) United States
(12) Patent Application Publication
(10) Pub. No.: US 2023/0331844 A1
(43) Pub. Date: Oct. 19, 2023

(54) ANTIBODY CONSTRUCTS TO TARGET T CELL RESPONSES TO SARS-COV PROTEIN EXPRESSING CELLS, THEIR DESIGN AND USES

(71) Applicant: Greffex, Inc., Houston, TX (US)

(72) Inventor: Uwe D. STAERZ, Denver, CO (US)

(21) Appl. No.: 18/025,684

(22) PCT Filed: Oct. 12, 2021
(86) PCT No.: PCT/US2021/054502

§ 371 Date: Mar. 10, 2023

Related U.S. Application Data
(60) Provisional application No. 63/090,557, filed on Oct. 12, 2020.

(51) Int. Cl. C07K 16/28; A61P 31/12; C07K 16/10

(52) U.S. Cl. CPC C07K 16/2809; C07K 16/283; A61P 31/12; C07K 16/1018

(57) ABSTRACT

The present disclosure provides a hybrid ligand molecule. The hybrid ligand molecule has two antibody combining sites. The first antibody combining site binds to an effector cell receptor complex structure of an effector cell. The second antibody combining site is a target cell-specific antibody combining site. The first and second antibody combining sites are linked.

### CROSS REFERENCE TO RELATED APPLICATIONS

This application claims priority to and is a non-provisional application of Provisional Application No. 63/090,557, filed on Oct. 12, 2020, which is incorporated herein by reference in its entirety.

### FIELD

The disclosure relates to ligand molecules, and, more particularly to ligand molecules comprising a T lymphocyte-activating agent linked to a viral target cell-associated antibody-combining site.

### BACKGROUND

Coronaviruses (CoVs) are classified into four genera: alpha-, beta-, gamma- and delta-coronaviruses. β-CoVs are enveloped, positive-strand RNA (30 kb) viruses capable of infecting mammals, generally bats and rodents, though many β-CoVs are known to infect humans as well. The virus enters the host cell via the angiotensin-converting enzyme 2 (ACE2). Of their four major structural proteins, the S protein mediates cell receptor binding. It is divided into the S1 and S2 chains which are separated by a furan cut site. The SARS receptor binding domain (RBD) is located in S1, and the membrane fusion section is located in S2. Other major proteins include the M, N and envelop (E) proteins.

Infections with CoVs in humans and animals commonly produce mild to moderate upper-respiratory tract illnesses of short duration. Exceptions are the Severe Acute Respiratory Syndrome (SARS-1), the Middle East Respiratory Syndrome (MERS) and the Wuhan-originating SARS-CoV-2 (SARS-2) (also referred to as COVID-19) that are characterized by severe and often lethal symptoms. The first cases of MERS were reported in Saudi Arabia in September 2012, with major eruptions in 2014 and 2015, followed by small seasonal outbreaks. 2,494 cases of confirmed MERS have so far been observed resulting in deaths in 858 patients (34.3% lethality; WHO). The first cases of SARS-2 infections were seen in December 2019. As of Apr. 16, 2020, there were an estimated 632,000 cases reported and an estimated 31,000 deaths in the United States alone, as reported by the Center for Disease Control (CDC), resulting in a 4.9% lethality. SARS-2 is highly infectious to humans, with an Ro estimated around 3 (Liu 2020). The World Health Organization (WHO) declared the SARS-2 worldwide pandemic a Global Health Emergency on Jan. 30, 2020.

In the United States, SARS-2 has been reported in all 50 states, Washington D.C., and at least 4 territories. Outbreaks in long-term care facilities and homeless shelters have emphasized the risk of exposure and infection in congregate settings. Person-to-person transmission either direct or through droplets is assumed to be the primary means of transmission of SARS-2.

While SARS-2 generally presents as a mild illness, with the most common symptoms being fever, cough or chest tightness, and dyspnea, the disease is most fatal for older and polymorbid patients. Severe complications include pneumonia, hypercoagulation, multiorgan dysfunction (including myocardial injury and kidney) and ultimately death. In children, multisystem inflammatory syndrome (MIS-C) is a serious condition in which some body parts, such as the heart, blood vessels, kidneys, digestive system, brain, skin or eyes, become inflamed.

Specific treatments for SARS-2 are not available but under investigation. The present recommendation is to observe patients with asymptomatic or mild illness. Given that a pulmonary disease can rapidly progress in patients with moderate disease, they should be closely monitored. Severe forms of the disease are associated with acute respiratory distress, virus-induced distributive shock, cardiac dysfunction, cytokine storm and also broad organ failures. Specific treatments for patients suffering from SARS-2 are still under clinical investigation with the antiviral Remdesivir (Gilead) showing promise in improving clinical outcome and being presently recommended for hospitalized patients. The question whether hydroxychloroquine has any use for SARS-2 treatment is still open.

CoVs induce both humoral and cellular immune responses. Animal, as well as clinical, studies demonstrate that SARS-1 and MERS infections raise potent neutralizing antibody responses against the S protein. Further, SARS-2 humoral responses similarly targeted the S protein with other antibodies binding to the M protein. The M protein also serves as a focus of CD8⁺ T cell responses. Anti-SARS-2 CD4⁺ T cells principally see both the N and the S antigen. Inactivated virus vaccines are inherently multivalent. They may provide stronger SARS-2 responses than single S protein vaccines. Animal studies have suggested that inactivated virus vaccines are prone to the induction of Th2-type possibly anti-N disease enhancing immune responses. Disease enhancement was also observed with S-based component vaccines, yet they were not evident with virally vectored anti-S vaccines. The FDA prefers SARS-2 vaccines that demonstrate Th1-type T cell polarization together with strong neutralizing antibodies.

Overall mutation rates of SARS-related (SARSr) viruses have been calculated at 0.1 mutations/generation. Minor changes in the S receptor binding domain of animal SARSr viruses can enhance binding to the human ACE2 and therefore facilitate a jump to the human population. Aligning S protein sequences reveals significant divergence throughout the gene with significant stable areas within the S2 region, whereas the M and N of the SARSr viruses show a significantly lower mutation rate overall.

The clearance of a virus infection involves a complex interaction by innate and adaptive immune responses. Infected cells release pro-inflammatory mediators, which can initiate host innate and adaptive immune responses. Specific immunity that is critical to resolving viral infections deploys two arms. In humoral immunity, B cells guided by CD4⁺ helper T (T_{H2}) cells produce antibodies. Neutralizing Abs inhibit viruses from engaging their cell surface receptors therefore preventing cell entry. Cell surface or pathogen bound Abs receptors therefore preventing cell entry. Cell surface or pathogen bound Abs can mediate cell lysis and phagocytosis through their constant (C) regions. Cellular immune responses are critical for an efficient clearance of viral infections. CD8⁺ cytotoxic T cells (CTL) play a crucial role in deleting the foci of viral replication and thus terminating the infectious process. Once they have recognized an infected cell, they kill it by the release of perform and granzymes creating cell membrane pores and triggering apoptotic pathways. CD4⁺ T_{H1} cells support virus clearance by driving inflammatory processes further broadening the immune response.

A T cell, or T lymphocyte, is a type of lymphocyte (a subtype of white blood cell) that plays a central role in cell-mediated immunity. T calls can be distinguished from other lymphocytes, such as B cells and actual killer cells, by the presence of a T-cell receptor on the cell surface. The several subsets of T cells each have a distinct function. The majority of T cells rearrange their alpha and beta chains on the cell receptor and are termed alpha beta T cells (αβ T cells) and are part of the adaptive immune system. Specialized gamma delta T cells, a small minority of T cells in the human body, have invariant T cell receptors with limited diversity, that can effectively present antigens to other T cells and are considered to be part of the innate immune system.

The category of effector T cell is a broad one that includes various T cell types that actively respond to a stimulus, such as co-stimulation. This includes helper, killer, regulatory, and potentially other T cell types. T helper cells (T_{H} cells) assist other white blood cells in immunologic processes, including maturation of B cells into plasma cells and memory B cells, and activation of cytotoxic T cells and macrophages. These cells are also known as CD4⁺ T cells because they express the CD4 glycoprotein on their surfaces. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate different types of immune responses. Signaling from the APC directs T cells into particular subtypes.

Cytotoxic T cells (T_{C} cells, CTLs, T-killer cells, killer T cells) destroy virus-infected cells and tumor cells, and are also implicated in transplant rejection. These cells are also known as CD8⁺ T cells since they express the CD9 glycoprotein at their surfaces. These cells recognize their targets by binding to antigen associated with MHC class I molecules, which are present on the surface of all nucleated cells. Through IL-10, adenosine, and other molecules secreted by regulatory T cells, the CD9⁺ cells can be inactivated to an anergic state, which prevents autoimmune diseases.

Natural killer T cells (NKT cells—not to be confused with natural killer cells of the innate immune system) bridge the adaptive immune system with the innate immune system. Unlike conventional T cells that recognize peptide antigens presented by major histocompatibility complex (MHC) molecules, NKT cells recognize glycolipid antigen presented by a molecule called CD1d. Once activated, these cells can perform functions ascribed to both Tₕ and Tc cells (i.e., cytokine production and release of cytolytic/cell killing molecules). They are also able to recognize and eliminate some tumor cells and cells infected with herpes viruses.

Gamma delta T cells (γδ T cells) represent a small subset of T cells that possess a distinct T cell receptor (TCR) on their surfaces. A majority of T cells have a TCR composed of two glycoprotein chains called α- and β-TCR chains. The antigenic molecules that activate γδ T cells are still widely unknown. However, γδ T cells are not MHC-restricted and seem to be able to recognize whole proteins rather than requiring peptides to be presented by MHC molecules on APCs. Some murine γδ T cells recognize MHC class IB molecules, though. Human Vγ0/Vδ2 T cells, which constitute the major γδ T cell population in peripheral blood, are unique in that they specifically and rapidly respond to a set of nonpeptidic phosphorylated isoprenoid precursors, collectively named phosphoantigens which are produced by virtually all living cells. The most common phosphoantigens from animal and human cells (including cancer cells) are isopentenyl pyrophosphate (IPP) and its isomer dimethylallyl pyrophosphate (DMPP). Many microbes produce the highly active compound hydroxy-DMAPP (HMB-PP) and corresponding mononucleotide conjugates, in addition to IPP and DMAPP. Plant cells produce both types of phosphoantigens. Drugs activating human Vγ9/Vδ2 T cells comprise synthetic phosphoantigens and aminobisphosphonates, which upregulate endogenous IPP/DMAPP.

The T lymphocyte activation pathway: T cells contribute to immune defenses in two major ways; some direct and regulate immune responses; others directly attack infected or cancerous cells. Activation of CD4⁺ T cells occurs through the simultaneous engagement of the T cell receptor and a co-stimulatory molecule (like CD29, or ICOS) on the T cell by the major histocompatibility complex (MHCII) peptide and co-stimulatory molecules on the APC. Activation of CD8⁺ T cells occurs through the simultaneous engagement of the T cell receptor and a co-stimulatory molecule (like CD28, or ICOS) on the T cell by the major histocompatibility complex (MHCI) peptide and co-stimulatory molecules on the APC. Both signals are required for production of an effective immune response; in the absence of co-stimulation, T-cell receptor signaling alone results in anergy. The signaling pathways downstream from co-stimulatory molecules usually engages the PI3K pathway generating PIP3 at the plasma membrane and recruiting PH domain containing signaling molecules like PDK1 that are essential for the activation of PKCθ, and eventual IL-2 production. Optimal CD8⁺ T cell response relies on CD4⁺ signaling. CD4⁺ cells are useful in the initial antigenic activation of naïve CD8 T cells, and sustaining memory CD8⁺ T cells in the aftermath of an acute infection. Therefore, activation of CD4⁺-T cells can be beneficial to the action of CD8⁺ T cells.

The first signaling is provided by binding of the T cell receptor to its cognate peptide presented on MHCII on an APC. MHCII is restricted to so-called professional antigen-presenting cells, like dendritic cells, B cells, and macrophages, to name a few. The peptides presented to CD8⁺ T cells by MHC class I molecules are 8-9 amino acids in length; the peptides presented to CD4⁺ cells by MHC class II molecules are longer, usually 12-25 amino acids in length, as the ends of the binding cleft of the MHC class II molecule are open. The second signal comes from co-stimulation, in which surface receptors on the APC are induced by a relatively small number of stimuli, usually products of pathogens, but sometimes breakdown products of cells, such as necrotic-bodies or heat shock proteins. The only co-stimulatory receptor expressed constitutively by naïve T cells is CD28, so co-stimulation for these cells comes from the CD80 and CD86 proteins, which together constitute the B7 protein, B7.1 and B7.2, respectively, on the APC. Other receptors are expressed upon activation of the T cell, such as OX40 and ICOS, but these largely depend upon CD28 for their expression. The second signal licenses the T cell to respond to an antigen. Without it, the T cell becomes anergic, and it becomes more difficult for it to activate in the future. This mechanism prevents inappropriate responses to self, as self-peptides will not usually be presented with suitable co-stimulation. Once a T cell has been appropriately activated (i.e., has received signal one and signal two), it alters its cell surface expression of a variety of proteins. Markers of T cell activation include CD69, CD71 and CD25 (also a marker for Treg cells) and HLA-DR (a marker of human T cell activation). CTLA-4 expression is also up-regulated on activated T cells, which in turn outcompetes CD28 for binding to the B7 proteins. This is a checkpoint mechanism to prevent over activation of the T cell. Activated T cells also change their cell surface glycosylation profile.

The T cell receptor exists as a complex of several proteins. The actual T cell receptor is composed of two separate peptide chains, which are produced from the independent T cell receptor alpha and beta (TCRα and TCRβ) genes. The other proteins in the complex are the CD3 proteins: CD3εγ and CD3εδ heterodimers and, most important, a CD3ζ homodimer, which has a total of six ITAM motifs. The ITAM motifs on the CD3 can be phosphorylated by Lck and in turn recruit ZAP-70. Lck and/or ZAP-70 can also phosphorylate the tyrosines on many other molecules, not least CD28, LAT and SLP-76, which allows the aggregation of signaling complexes around these proteins. Phosphorylated LAT recruits SLP-76 to the membrane, where it can then bring in PLC-γ, VAV1, 1tk and potentially PI3K. PLC-γ cleaves PI(4,5)P2 on the inner leaflet of the membrane to create the active intermediaries diacylglycerol (DAG), inositol-1,4,5-trisphosphate (IP3); PI3K also acts on PIP2, phosphorylating it to produce phosphatidylinositol-3,4,5-trisphosphate (PIP3). DAG binds and activates some PKCs. Most important in T cells is PKCθ, critical for activating the transcription factors NF-κB and AP-1. IP3 is released from the membrane by PLC-γ and diffuses rapidly to activate calcium channel receptors on the ER, which induces the release of calcium into the cytosol. Low calcium in the endoplasmic reticulum causes STIM1 clustering on the ER membrane and leads to activation of cell membrane CRAC channels that allows additional calcium to flow into the cytosol from the extracellular space. This aggregated cytosolic calcium binds calmodulin, which can then activate calcineurin. Calcineurin, in turn, activates NFAT, which then translocates to the nucleus. NFAT is a transcription factor that activates that transcription of a pleiotropic set of genes, most notable, IL-2, a cytokine that promotes long-term proliferation of activated T cells. PLCγ can also initiate the NF-κB pathway. DAG activates PKCθ, which then phosphorylates CARMA1, causing it to unfold and function as a scaffold. The cytosolic domains bind an adapter BCL10 via CARD (Capase activation and recruitment domains) domains' that then binds TRAF6, which is ubiquitinated a K63. This form of ubiquitination does not lead to degradation of target proteins. Rather, it serves to recruit NEMO, IKKα and -β, and TAB1-2/TAK1. TAK 1 phosphorylates IKK-β, which then phosphorylates IκB allowing for K48 ubiquitination: leads to proteosomal degradation. Rel A and p50 can then enter the nucleus and bind the NF-κB response element. This coupled with NFAT signaling allows for complete activation of the IL-2 gene.

During their development immature T lymphocytes that mature into CD4⁺ and CD8⁺ T lymphocytes are selected for their ability to recognize foreign antigens in conjunction with the set of class I and class II MHC molecules present in the respective human or animal. Immature T lymphocytes that cannot use the present set of MHC molecules do not mature during this positive selection process in the thymus. In another process immature T lymphocytes that recognize peptides derived from self antigens present in the respective human or animal are not allowed to develop into mature T lymphocytes. This process is called negative selection. As a result of these two processes, the repertoire of mature T cells present in a human or an animal can only recognize peptides derived from antigens in conjunction with an MHC molecule present within the set of MHC molecules present during their maturation. As different humans and animals generally carry different sets of MHC molecules, their ab T cells will not function upon transfer into a new host unless a given ab T cell finds a MHC molecule in the host identical or closely related to the one, by which it was positively selected in the donor. This MHC restricted recognition by ab T cells makes it difficult, if not impossible, to transfer ab T cells and thus their effector function from one human or animal to another human or animal.

An antibody (Ab), also known as an immunoglobulin (Ig), is a Y-shaped protein produced mainly by plasma cells that is used by the immune system to identify and neutralize pathogens such as bacteria and viruses. An antibody recognizes molecules, such as, but not limited to, proteins or carbohydrates, called an antigen, via the Fab's variable region. Each tip of the “Y” of an antibody contains a paratope (analogous to a lock) that is specific for one particular epitope (similarly analogous to a key) on an antigen, allowing these two structures to bind together with precision. An epitope is defined as a region or a domain of antigen, to which the antibody hypervariable region or paratope binds. The ability of an antibody to communicate with the other components of the immune system is mediated via its Fc region (located at the base of the “Y”), which contains a conserved glycosylation site involved in these interactions. The production of antibodies is the main function of the humoral immune system. Antibodies are secreted by B cells of the adaptive immune system, mostly by differentiated B cells called plasma cells. Antibodies can occur in two physical forms, a soluble form that is secreted from the cell to be free in the blood plasma, and a membrane-bound form that is attached to the surface of a B cell and is referred to as the B-cell receptor (BCR). The BCR is found only on the surface of B cells and facilitates the activation of these cells and their subsequent differentiation into either antibody factories called plasma cells or memory B cells that will survive in the body and remember that same antigen so the B cells can respond faster upon future exposure. Soluble antibodies are released into the blood and tissue fluids, as well as many secretions to continue to survey for invading microorganisms. B cells and plasma cells that produce a given antibody, can be immortalized by fusing these cells to an immortalized cell. The resulting fusion product, called hybridoma, can be indefinitely propagated and can be used to produce the respective single antibody in large amounts. Alternatively, the B cells and plasma cells can be immortalized by their infection with certain viruses, such as, but not limited to, an Epstein Barr Virus (EBV) or their transfection with immortalizing genes.

Antibodies are glycoproteins belonging to the immunoglobulin superfamily. They constitute most of the gamma globulin fraction of the blood proteins. They are typically made of basic structural units—each with two large heavy chains and two small light chains. There are several different types of antibody heavy chains that define the five different types of crystallizable fragments (Fc) that may be attached to the antigen-binding fragments. The five different types of Fc regions allow antibodies to be grouped into five isotypes. Each Fc region of a particular antibody isotype is able to bind to its specific Fc receptor (except for IgD, which is essentially the BCR), thus allowing the antigen-antibody complex to mediate different roles depending on which FcR it binds. The ability of an antibody to bind to its corresponding FcR is further modulated by the structure of the glycan(s) present at conserved sites within its Fc region. The ability of antibodies to bind to FcRs helps to direct the appropriate immune response for each different type of foreign object they encounter. For example, IgE is responsible for an allergic response consisting of mas cell degranulation and histamine release. IgE's Fab paratope binds to allergic antigen, for example, house dust mite particles, while its Fc region binds to Fc receptor E. The allergen-IgE-FcRε interaction mediates allergic signal transduction to induce conditions such as asthma.

Antibody and antigen interact by spatial complementarity (lock and key). The molecular forces involved in the Fab-epitope interaction are weak and non-specific—for example electrostatic forces, hydrogen bonds, hydrophobic interactions, and van der Waals forces. This means binding between antibody and antigen is reversible, and the antibody's affinity towards an antigen is relative rather than absolute. Relatively weak binding also means it is possible for an antibody to cross-react with different antigens of different relative affinities. Haptens are small molecules that provide no immune response by themselves, but can still be recognized by an antibody.

Antibodies are heavy (˜150 kDa) globular plasma proteins. They have sugar chains (glycans) added to conserved amino acid residues. In other words, antibodies are glycoproteins. Among other things, the expressed glycans can modulate an antibody's affinity for its corresponding FcR(s). The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds. Each chain is composed of structural domains called immunoglobulin domains. These domains contain about 70-110 amino acids and are classified into different categories (for example, variable or IgV, and constant or IgC) according to their size and function. They have a characteristic immunoglobulin fold in which two beta sheets create a “sandwich” shape, held together by interactions between conserved cysteines and other charged amino acids.

There are five types of mammalian Ig heavy chain denoted by the Greek letters α, β, ε, γ, and μ. The type of heavy chain present defines the class of antibody; these chains are found in IgA, IgD, IgE, IgG and IgM antibodies, respectively. Distinct heavy chains differ in size and composition; α and γ contain approximately 450 amino acids, whereas p and E have approximately 550 amino acids.

The domains and structures of a typical antibody are:
Fab region;
Fc region;
Heavy chain (blue) with one variable (V_{H}) domain followed by a constant domain (C_{H}1),
a hinge region, and two more constant (C_{H}2 and C_{H}3) domains;
Light chain with one variable (V_{L}) and one constant (C_{L}) domain;
Antigen binding site (paratope); and
Hinge regions.

The heavy chain has two regions, the constant region and the variable region. The constant region is identical in all antibodies of the same isotype, but differs in antibodies of different isotypes. Heavy chains γ, α, and δ have a constant region composed of three tandem (in a line) Ig domains, and a hinge region for added flexibility; heavy chains p and E have a constant region composed of four immunoglobulin domains. The variable region of the heavy chain differs in antibodies produced by different B cells, but is the same for all antibodies produced by a single B cell or B cell cone. The variable region of each heavy chain is approximately 110 amino acids long and is composed of a single Ig domain.

In mammals there are two types of immunoglobulin light chain, which are called lambda (λ) and kappa (κ). A light chain has two successive domains: one constant domain and one variable domain. The approximate length of a light chain is 211 to 217 amino acids. Each antibody contains two light chains that are always identical; only one type of light chain, κ or λ, is present per antibody in mammals. Other types of light chains, such as the iota (I) chain, are found in other vertebrates like sharks (Chondrichthyes) and bony fishes (Teleostei).

Some parts of an antibody have the same functions. The arms of the Y, for example, contain the sites that can bind to antigens (in general, identical) and, therefore, recognize specific foreign objects. This region of the antibody is called the Fab (fragment, antigen-binding) region. It is composed of one constant and one variable domain from each heavy and light chain of the antibody. The paratope is shaped at the amino terminal end of the antibody monomer by the variable domains from the heavy and light chains. The variable domain is also referred to as the Fv region and is the most important region for binding to antigens. To be specific, variable loops of β-strands, three each on the light (V_{L}) and heavy (V_{H}) chains are responsible for binding to the antigen. These loops are referred to as the complementarity determining regions (CDRs).

The base of the Y plays a role in modulating immune cell activity. This region is called the Fc (Fragment, crystallizable) region, and is composed of two heavy chains that contribute two or three constant domains depending on the class of the antibody. Thus, the Fc region ensures that each antibody generates an appropriate immune response for a given antigen, by binding to a specific class of Fc receptors, and other immune molecules, such as complement proteins. By doing this, it mediates different physiological effects including recognition of opsonized particles (binding to FcγR), lysis of cells (binding to complement), and degranulation of mas cells, basophils, and eosinophils (binding to FcεR).

In summary, the Fab region of the antibody determines antigen specificity while the Fc region of the antibody determines the antibody's class effect. Since only the constant domains of the heavy chains make up the Fc region of an antibody, the classes of heavy chain in antibodies determine their class effects. Possible classes of heavy chains in antibodies include alpha, gamma, delta, epsion, and mu, and they define the antibody's isotypes IgA, G, D, E and M, respectively. This infers different isotypes of antibodies have different class effects due to their different Fc regions binding and activating different types of receptors. Possible class effects of antibodies include: Opsonisation, agglutination, haemolysis, complement activation, mast cell degranulation, and neutralization (though this class effect may be mediated by the Fab region rather than the Fc region). It also implies that Fab-mediated effects are directed at microbes or toxins, whilst Fc mediated effects are directed at effector cells or effector molecules.

To combat pathogens that replicate outside cells, antibodies bind to pathogens to link them together, causing them to agglutinate. Since an antibody has at least two paratopes, it can bind more than one antigen by binding identical epitopes carried on the surfaces of these antigens. By coating the pathogen, antibodies stimulate effector functions against the pathogen in cells that recognize their Fc region. Those cells that recognize coated pathogens have Fc receptors, which, as the name suggests, interact with the Fc region of IgA, IgG, and IgE antibodies. The engagement of a particular antibody with the Fc receptor on a particular cell triggers an effector function of that cell; phagocytes will phagocytose, mast cells and neutrophils will degranulate, natural killer cells will release cytokines and cytotoxic molecules; that will ultimately result in destruction of the invading microbe. The activation of natural killer cells by antibodies initiates a cytotoxic mechanism known as antibody-dependent cell-mediated cytotoxicity (ADCC)—this process may explain the efficacy of monoclonal antibodies used in biological therapies against cancer. The Fc receptors are isotype-specific, which gives greater flexibility to the immune system, invoking only the appropriate immune mechanisms for distinct pathogens.

Certain cells can recognize coated pathogens via their Fc receptors, which, as the name suggests, interact with the Fc region of IgA, IgG, and IgE antibodies. The engagement of a particular antibody with the Fc receptor on a particular cell triggers an effector function of that cell; phagocytes will phagocytose, mast cells and neutrophils will degranulate, natural killer cells will release cytokines and cytotoxic molecules; that will ultimately result in destruction of the invading microbe. The activation of natural killer cells by antibodies initiates a cytotoxic mechanism known as antibody-dependent cell-mediated cytotoxicity (ADCC)—this process may explain the efficacy of monoclonal antibodies used in biological therapies against cancer. The Fc receptors are isotype-specific, which gives greater flexibility to the immune system, invoking only the appropriate immune mechanisms for distinct pathogens.

An Fc receptor is a protein found on the surface of certain cells—including, among others, B lymphocytes, follicular dendritic cells, natural killer cells, macrophages, neutrophils, eosinophils, basophils, human platelets, and mast cells—that contribute to the protective functions of the immune system. Its name is derived from its binding specificity for a part of an antibody known as Fc (Fragment, crystallizable) region. Fc receptors bind to antibodies that are attached to infected cells or invading pathogens. Their activity stimulates phagocytic or cytotoxic cells to destroy microbes, or infected cells by antibody-mediated phagocytosis or antibody-dependent cell-mediated cytotoxicity (ADCC).

All of the Fcγ receptors (FcγR) belong to the immunoglobulin superfamily and are the most important Fc receptors for inducing phagocytosis of opsonized (marked) microbes. This family includes several members, FcγRI (CD64), FcγRIIA (CD32), FcγRIIB (CD32), FcγRIIIA (CD16a), FcγRIIIB (CD16b), which differ in their antibody affinities due to their different molecular structure. For instance, FcγRI binds to IgG more strongly than FcγRII or FcγRIII does. FcγRI also has an extracellular portion composed of three immunoglobulin (Ig)-like domains, one more domain than FcγRII or FcγRIII has. This property allows FCγRI to bind a sole IgG molecule (or monomer), but all Fcγ receptors must bind multiple IgG molecules within an immune complex to be activated.

There are principally two strategies to circumvent the limitations of the MHC restricted recognition by T lymphocytes, (1) chimeric T cell receptor: the engineering of the TCR complex in a way that the specificity of an antibody, such as a broadly reactive anti-viral antibody, is grafted onto the members of the TCR complex or (2) hybrid antibody: the use of an antibody constructs that links the variable domain of an antibody, such as one of a broadly reactive anti-viral antibody, to antibody variable regions that bind to a member or members of the TCR complex alone or also with a T cell accessory molecule.

Artificial T cell receptors (also known as chimeric T cell receptors, chimeric immunoreceptors, chimeric antigen receptors (CARs)) are engineered receptors, which graft an antibody specificity onto TCR complex. Typically, these receptors are used to graft the specificity of a monoclonal antibody onto a T cell; with transfer of their coding sequence facilitated by retroviral vectors. The most common form of CARs are fusions of single-chain variable fragments (scFv) derived from monoclonal antibodies, fused to CD3-zeta transmembrane and endodomain. An example of such construct is 14g2a-Zeta, which is a fusion of a scFv derived from hybridoma 14g2a (which recognizes disialoganglioside GD2).

The variable portions of an immunoglobulin heavy and light chain are fused by a flexible linker to form an scFv. This scFv is preceded by a signal peptide to direct the nascent protein to the endoplasmic reticulum and subsequent surface expression (cloven). A flexible spacer allows the scFv to orient in different directions to enable antigen binding. The transmembrane domain is a typical hydrophobic alpha helix usually derived from the original molecule of the signaling endodomain that protrudes into the cell and transmits the desired signal. ScFv/CD3-zeta hybrids result in the transmission of a zeta signal in response to recognition by the scFv of its target.

The most commonly used endodomain component is CD3-zeta which contains 3 ITAMs. This transmits an activation signal to the T cell after the antigen is bound. CD3-zeta may not provide a fully competent activation signal and co-stimulatory signaling is needed. For example, chimeric CD29 and OX40 can be used with CD3-Zeta to transmit a proliferative/survival signal or all three can be used together. First generation CARs typically had the intracellular domain from the CD3 ζ-chain, which is the primary transmitter of signals from endogenous TCRs. Second generation CARs add intracellular signaling domains from various costimulatory protein receptors (e.g., CD29, 41BB, ICOS) to the cytoplasmic tail of the CAR to provide additional signals to the T cell. Preclinical studies indicated that the second generation improves the antitumor activity of T cells. More recent, third generation CARs combine multiple signaling domains, such as CD3z-CD28-41BB or CD3z-CD28-OX40, to augment potency.

A hybrid antibody (HAb), such as a bispecific monoclonal antibody or multispecific monoclonal antibody, is an artificial protein that is composed of fragments of two (or more) different monoclonal antibodies and consequently binds to two (or more) different types of antigens. The most widely used application of this approach is in cancer immunotherapy, where a HAb is engineered to simultaneously bind to a cytotoxic cell (using a receptor, such as component of the T cell receptor complex, for instance CD3, or of an Fc receptor complex) and a target like a tumor cell to be destroyed.

HAbs have been produced in different ways. A first-generation of HAbs consisted of two heavy and two light chains, one each from two different antibodies. The two Fab regions (the arms) are directed against two antigens. The Fc region (the foot) is made up from the two heavy chains and forms the third binding site; hence the name. Such antibodies could be produced by fusing the two B cell hybridomas that produced the two antibodies of interest. These so-called quadromas then released the antibodies, from which the HAb in question could be purified. Alternatively the different antibody chains were genetically cloned into expression vectors that were then transfected into a production cell.

Other types of bispecific antibodies have been designed. They include chemically linked Fabs, consisting only of the Fab regions, and various types of bivalent and trivalent single-chain variable fragment (scFvs), fusion proteins mimicking the variable domains of two antibodies. The furthest developed of these newer formats are the bi-specific T-cell engagers and mAb2's, antibodies engineered to contain an Fcab antigen-binding fragment instead of the Fc constant region.

For instance, of the two paratopes that form the tops of the variable domains, one can be directed against a tumor antigen and the other against an T-lymphocyte antigen like CD3. In some cases, an Fc region can also bind to a cell that expresses Fc receptors, like a macrophage, a natural killer cell or a dendritic cell.

This family includes several members, FcγRI (CD674), FcγRIIA (CD32), FcγRIIB (CD32), FcγRIIIA (CD16a), RcγRIIIB (CD16b), which differ in their antibody affinities due to their different molecular structure. For instance, FcγRI binds to IgG more strongly than RcγRII or FcγRIII does. FcγRI also has an extracellular portion composed of three immunoglobulin (Ig)-like domains, one more domain than FcγRII or FcγRIII has. This property allows FcγRI to bind a sole IgG molecule (or monomer), but all Fcγ receptors must bind multiple IgG molecules within an immune complex to be activated.

Fc receptors (FcR) belong to the immunoglobulin superfamily. It will be possible to graft an antibody specificity on these receptors by (1) chimeric Fc receptor: the engineering of the Fc receptor complex in a way that the specificity of an antibody, such as a broadly reactive anti-viral antibody, is grafted onto the members of the Fc receptor complex or (2) hybrid antibody: the use of an antibody constructs that links the variable domain of an antibody, such as one of a broadly reactive anti-viral antibody, to antibody variable regions that bind to a member or members of the Fc receptor complex alone or also with an accessory molecule.

### SUMMARY

The present disclosure provides a hybrid ligand molecule. In accordance with embodiments of the present disclosure, a hybrid ligand molecule comprises a first antibody combining site that binds to an effector cell receptor complex structure of an effector cell linked to a second antibody combining site which is a target cell-specific antibody combining site.

In an embodiment, the effector cell is a T lymphocyte and the effector cell receptor complex structure is a T cell receptor complex structure. In a further embodiment, the first antibody combining site is directed to a T cell antigen receptor on a surface of the T lymphocyte. In yet a further embodiment, the first antibody combining site is directed to a CD3 complex on a surface of the T lymphocyte.

In an embodiment, the second antibody combining site binds to a protein encoded by a virus which is expressed on a surface of the target cell. In a further embodiment, the second antibody combining site is directed to a viral protein encoded within a coronavirus and expressed on the surface of the target cell. In still another embodiment, the first antibody combining site binds to a T cell receptor complex structure and is capable of activating T lymphocytes.

The present disclosure provides a hybrid ligand molecule. In accordance with embodiments of the present disclosure, a hybrid ligand molecule comprises a first antibody combining site that binds to an activating cell surface receptor on an effector cell.

In an embodiment, the effector cell is selected from the group consisting of T cells, natural killer cells, phagocytotic cells, and combinations thereof. In another embodiment, the effector cell is selected from the group consisting of alpha beta T cells, gamma delta T cells, natural killer cells, and phagocytotic cells. In another embodiment, the activating cell surface receptor is selected from the group consisting of a CD3 complex, FcγRI (CD64), FcγRIIA (CD32), FcγRIIB (CD32), FcγRIIIA (CD16a), FcγRIIIB (CD16b), and combinations thereof. In still another embodiment, the activating cell surface receptor is selected from the group consisting of FcγRI (CD64), FcγRIIA (CD32), FcγRIIB (CD32), FcγRIIIA (CD16a), FcγRIIIB (CD16b), and combinations thereof.

The present disclosure provides a hybrid ligand molecule. In accordance with embodiments of the present disclosure, a hybrid ligand molecule comprises a plurality of different, linked antibody combining sites, wherein at least a first of the plurality binds to a T cell receptor complex structure and at least a second of the plurality binds to a target cell-specific antigen.

The present disclosure provides a composition including hybrid ligand molecules. In accordance with embodiments of the present disclosure, the composition includes hybrid ligand molecules dispersed in a physiologically tolerable diluent, said hybrid ligand molecules comprising a plurality of different, linked antibody combining sites, wherein at least a first of the plurality binds to a T cell receptor complex structure and at least a second of the plurality binds to a target cell-specific antigen, wherein, when contacted in an effective amount in vitro with target cells in the presence of an exogenously supplied source of cytotoxic effector T lymphocytes, the fluid induces lysis of the target cells by said cytotoxic effector T lymphocytes.

The present disclosure provides a method of killing infected cells. In accordance with embodiments of the present disclosure, the method of killing infected cells comprises (a) providing a composition containing a unit dose of hybrid ligand molecules dispersed in a physiologically tolerable diluent, the hybrid ligand molecules comprising a first antibody combining site linked to a second antibody combining site, wherein the first antibody combining site binds to an effector cell receptor complex structure and the second antibody combining site binds to a virus specific antigen, and wherein the composition induces destruction of the virus infected cells by an effector cell that reacts with cells that bear the virus specific antigen; (b) contacting infected cells that bear the virus specific antigen with the composition in the presence of a source of effector cells whose production is activated by the first antibody combining site, wherein the composition is present in an amount sufficient to effect binding to the cytotoxic effector T lymphocytes and to the infected cells; and (c) maintaining the contacting for a time period sufficient (i) for the second antibody combining site to bind to the virus specific antigen and (ii) for the first antibody combining site to bind to and activate production of effector cells, wherein the produced effector cells react with the cells bearing the specific antigen.

In another embodiment, the infected cells are tumor cells. In a further embodiment, the effector cells are T cells. In yet another embodiment, the T cells are phagocytotic cells. In still a further embodiment, the method further includes the step of periodically repeating steps (a)-(c) until substantially all of the infected cells have been killed.

### BRIEF DESCRIPTION OF THE DRAWINGS

FIG. 1 is a diagrammatic presentation of an IgG antibody denoting domains of the Ig heavy and light chains and fragments of the antibody;

FIG. 2a is a diagrammatic presentation of a TCR receptor complex;

FIGS. 2b-d are examples of chimeric antibody/TCR constructs;

FIG. 3a is a diagrammatic presentation of the hybrid antibody construct bispecific Ab based on the reassembly of F(ab′)/F(ab) fragments;

FIG. 3b is a diagrammatic presentation of the hybrid antibody construct bispecific Ab as ‘complete’ Ig molecule;

FIG. 3c is a diagrammatic presentation of the hybrid antibody construct bispecific Ab composed of variable domains (Fv);

FIG. 3d is a diagrammatic presentation of the hybrid antibody construct bispecific Ab composed of variable domains (Fv) in a Bite configuration;

FIG. 3e is a diagrammatic presentation of the hybrid antibody construct trispecific Ab composed of variable domains (Fv) in a Trite configuration;

FIG. 3f is a diagrammatic presentation of the hybrid antibody construct tetraspecific Ab composed of variable domains (Fv) in a Qite configuration;

FIG. 3g is a diagrammatic presentation of the hybrid antibody construct trispecific Ab based on the reassembly of modified F(ab′) fragments in a Tribody configuration;

FIG. 3h is a diagrammatic presentation of the hybrid antibody construct trispecific Ab as ‘complete’ Ig molecule with a ‘Bite’ configuration arm in a TriAntibody configuration;

FIG. 3i is a diagrammatic presentation of the hybrid antibody construct tetraspecific Ab based on the reassembly of two modified F(ab′) fragments in ‘Bite’ configurations in a Quadbody configuration;

FIG. 3j is a diagrammatic presentation of the hybrid antibody construct tetraspecific Ab as ‘complete’ Ig molecule with two ‘Bite’ configuration arms in a QuadAntibody configuration;

FIGS. 4a and 4b are the graphical results of Experiment 1 described in the present application;

FIGS. 5a-h are diagrammatic presentations of a HAb that incorporates an anti-TCR complex antibody and a broadly reactive anti-viral antibody, and a HAb that incorporates an anti-TCR complex antibody and an anti-accessory molecule antibody and a broadly reactive anti-viral antibody; and

FIGS. 6a-f are diagrammatic presentations of chimeric TCRs that incorporate a hypervariable region of an anti-viral antibody.

### DETAILED DESCRIPTION

Before any embodiments of the present disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is understood that the phraseology and terminology used herein is for the purpose of description and should not be regarded as limiting. The use of “including,” “comprising” or “having” and variations thereof herein is meant to encompass the items listed thereafter and equivalents thereof as well as additional items. The use of “including essentially” and “consisting essentially of” and variations thereof herein is meant to encompass the items listed thereafter, as well as equivalents and additional items provided such equivalents and additional items to not essentially change the properties, use or manufacture of the whole. The use of “consisting of” and variations thereof herein is meant to include the items listed thereafter and only those items.

With reference to the drawings, like numbers refer to like elements throughout. It will be understood that, although the terms first, second, etc., may be used herein to describe various elements, components, regions, and/or sections, these elements, components, regions and/or sections should not be limited by these terms. These terms are used only to distinguish one element, component, region and/or section from another element, component, region and/or section. Thus, a first element, component, region or section could be termed a second element, component, region or section without departing from the disclosure.

The numerical ranges in this disclosure are approximate, and thus may include values outside of the range unless otherwise indicated. Numerical ranges include all values from and including the lower and the upper values (unless specifically stated otherwise), in increments of one unit, provided that there is a separation of at least two units between any lower value and any higher value. As an example, if a compositional, physical or other property, such as, for example, amount of a component by weight, etc., is from 10 to 100, it is intended that all individual values, such as 10, 11, 12, etc., and sub ranges, such as 10 to 44, 55 to 70, 97 to 100, etc., are expressly enumerated. For ranges containing explicit values (e.g., a range from 1, or 2, or 3 to 5, or 6, or 7), any subrange between any two explicit values is included (e.g., the range 1-7 above includes subranges 1 to 2; 2 to 6; 5 to 7; 3 to 7; 5 to 6, etc.). For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure.

Spatial terms, such as “beneath,” “below,” “lower,” “above,” “upper,” and the like, may be used herein for ease of description to describe one element's or feature's relationship to another element(s) or feature(s) as illustrated in the figures. It will be understood that the spatially relative terms are intended to encompass different orientations depending on the orientation in use or illustration. For example, if a device in the figures is turned over, elements described as “below” or “beneath” other elements or features would then be oriented “above” the other elements or features. Thus, the exemplary term “below” can encompass both an orientation of above and below. A device may be otherwise oriented (rotated 900 or at other orientations) and the spatially relative descriptors used herein interpreted accordingly.

As used herein, the term “and/or” includes any and all combinations of one or more of the associated listed items. For example, when used in a phrase such as “A and/or B,” the phrase “and/or” is intended to include both A and B; A or B; A (alone); and B (alone). Likewise, the term “and/or” as used in a phrase such as “A, B and/or C” is intended to encompass each of the following embodiments” A, B and C; A, B, or C; A or C; A or B; B or C; A and C; A and B; B and C; A (alone); B (alone); and C (alone).

The embodiments disclosed herein relate to incorporation of variable regions of broadly reactive anti-viral antibodies into chimeric TCRs, Fc receptors and multimeric antibody constructs that target TCRs or Fc receptors.

Unless defined otherwise, all technical and scientific terms used herein have the same meaning as commonly understood by one of ordinary skill in the art to which this disclosure belongs. Generally, the nomenclature used herein and the laboratory procedures in cell culture, molecular genetics, and nucleic acid chemistry and hybridization described below are those well-known and commonly employed in the art. Standard techniques are used for recombinant nucleic acid methods, polynucleotide synthesis, and microbial culture and transformation (e.g., electroporation, lipofection). Generally, enzymatic reactions and purification steps are performed according to the manufacturer's specifications. The techniques and procedures are generally performed according to conventional methods in the art and various general references (see generally, Sambrook et al. Molecular Cloning: a Laboratory Manual, 2d ed. (1989) Cold Spring Harbor Laboratory Press, Cold Spring Harbor, N.Y., which is incorporated herein by reference), which are provided throughout this document. Units, prefixes, and symbols may be denoted in their SI accepted form. Unless otherwise indicated, nucleic acids are written left to right in 5′ to 3′ orientation; amino acid sequences are written left to right in amino to carboxyl orientation, respectively. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5.sup.th edition, 1993).

As employed throughout the disclosure, the following terms, unless otherwise indicated, shall be understood to have the following meanings and are more fully defined by reference to the specification as a whole.

By “antigen” is meant a molecule, which contains one or more epitopes that will stimulate a host's immune system to make a cellular antigen-specific immune response, or a humoral antibody response. Thus, antigens include proteins, polypeptides, antigenic protein fragments, oligosaccharides, polysaccharides, and the like. Furthermore, the antigen can be derived from any known virus, bacterium, parasite, plants protozoans, or fungus, and can be a whole organism. The term also includes tumor antigens. Similarly, an oligonucleotide or polynucleotide which expresses an antigen, such as in DNA immunization applications, is also included in the definition of antigen. Synthetic antigens are also included, for example polyepitopes, flanking epitopes, and other recombinant or synthetically derived antigens (Bergmann et al. (1993) Eur. J. Immunol. 23:2777 2781; Bergmann et al. (1996) J. Immunol. 157:3242 3249; Suhrbier, A. (1997) Immunol. And Cell Biol. 75:402 408; Gardner et al. (1998) 12th World AIDS Conference, Geneva, Switzerland, Jun. 28-Jul. 3, 1998).

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A transcription termination sequence may be located 3′ to the coding sequence. Transcription and translation of coding sequences are typically regulated by “control elements,” including, but not limited to, transcription promoters, transcription enhancer elements, Shine and Delagamo sequences, transcription termination signals, polyadenylation sequences (located 3′ to the translation stop codon), sequences for optimization of initiation of translation (located 5′ to the coding sequence), and translation termination sequences.

The term “construct” refers to a genetic composition that codes for the expression of proteins.

T lymphocytes are a population of white cells in the body of humans and animals that carry a T cell receptor.

The term “T cell receptor (TCR)” refers to a receptor composed to two “variable chains” expressed in T lymphocytes that is used by T lymphocytes to recognize antigens. The term “TCR” refers to a TCR that is composed of two TCR chains denoted as alpha- and beta-chains.

The term “CD3” refers to a set of genes and proteins denoted g, d, e and z that are linked to the TCR and its ability to receive and transfer a biological trigger.

The term “TCR complex” refers to a cell surface complex found on T lymphocytes that is principally composed of a TCR and CD3 proteins.

The term “accessory molecules” refers to cell surface molecules found on lymphocytes that enable or enhance their activation or deactivation. They include, but are not limited to CD4, CD9, CD28, LFA-1, 4-1BB and OX40.

The term “B lymphocyte” refers to a population of white cells in humans and animals that have the ability to produce immunoglobulins.

The term “plasma cells” refers to a population of B lymphocytes that have the ability to produce immunoglobulins.

The terms “immunoglobulin,” “Ig” or “antibody” refer to proteins that are found in the bodies of humans and animals that have the ability to specifically bind to antigens. An “antibody combining site” or “paratope” refers to the portion of an antibody which binds to another molecule, e.g., antigen, T cell or TCR complex, etc.

The terms “hypervariable region” or “hypervariable domain” refer to an area within an antibody that is used for specific binding.

The term “heavy chain” refers to the larger protein chain found in an antibody. The term “light chain” refers to the smaller protein found in an antibody.

The term “Fc” refers to a non-variable section of an antibody composed of nonvariable sections of the heavy chain that can activate certain immune function, such as complement activation, or mediate the binding of the antibody to cell surface receptors called “Fc receptors” or “FcR.”

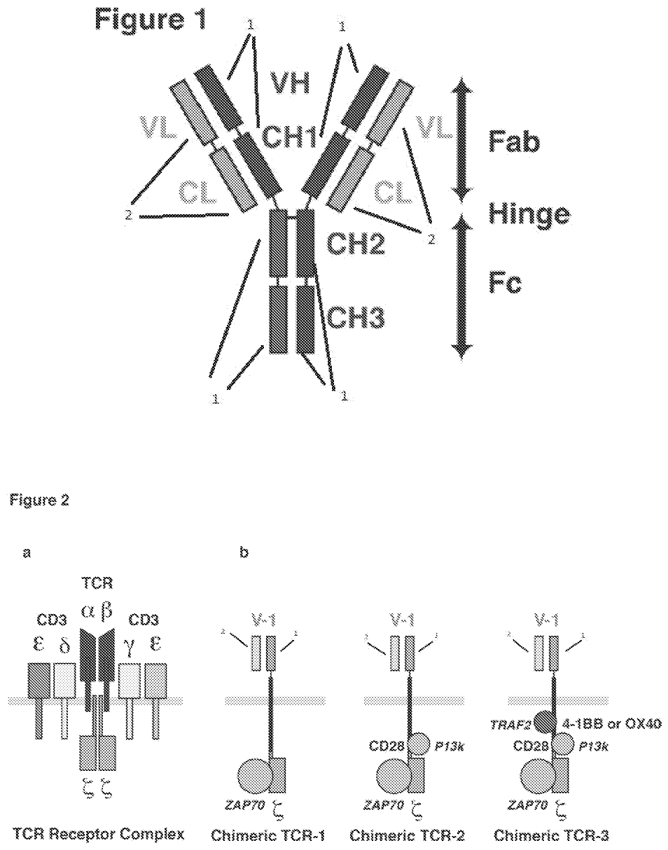

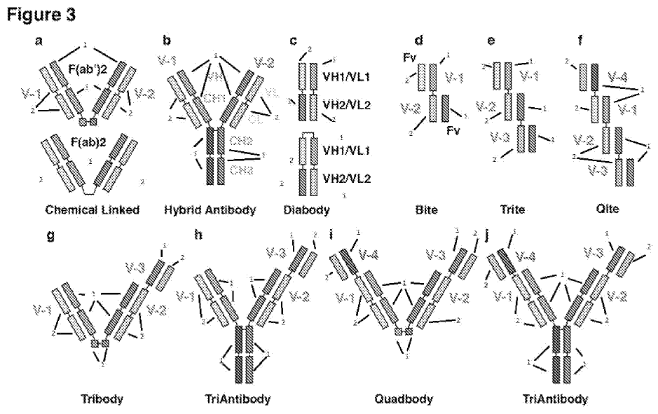

The term “Fab” refers to a section of an antibody that is composed of the hypervariable region and the first constant region of an antibody (as depicted in FIGS. 1 and 3) and the term “F(ab)′2” refers to a section of an antibody that is composed of the hypervariable region and the first constant region of an antibody together with fragments of the second constant region of the heavy chain so that a dimeric molecule is maintained (as depicted in FIGS. 1 and 3).

The terms “Fc receptor” or “FcR” refer to a cell surface receptor on certain white cells that have the ability to bind the Fc region of an antibody. The family of Fc receptors includes several members, FcγRI (CD64), FcγRIIA (CD32), FcγRIIB (CD32), FcγRIIIA (CD16a), FcγRIIIB (CD16b), which differ in their antibody affinities due to their different molecular structure.

The term “immune accessory cell” refers to certain white cells, such as, but not limited to, dendritic cells and macrophages that have the ability to interact with immune cells, such as, but not limited to T lymphocytes and/or B lymphocytes.

The terms “dendritic cells”, “macrophages,” “granulocytes,” “monocytes” and “neutrophiles” refer to populations of white cells found in humans and animals.

The terms “hybrid antibody” or “HAb” refer to an antibody whose structure has been modified so that it carries more than one hypervariable region.

The terms “bispecific antibody” or “BAb” refer to a HAb that has been modified so that it carries two different hypervariable regions.

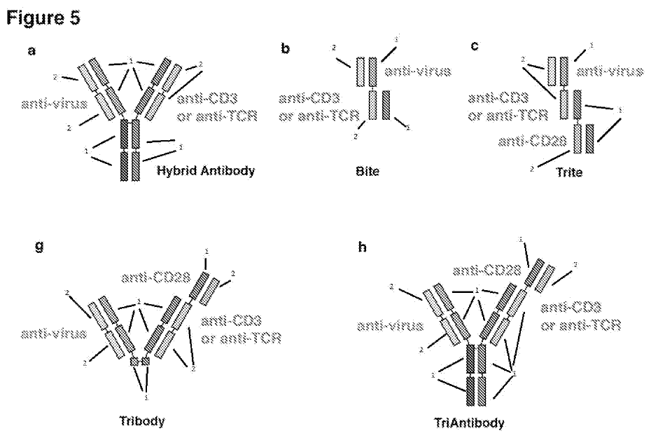

The terms “diabody,” “bite,” trite,” “qite,” “tribody,” “triantibody,” “quadbody,” and “triantibody” describe different versions of HAbs as exemplified in FIGS. 3 and 5.

The term “chimeric TCR” refers to a construct composed of components of the TCR complex that have been genetically modified so that antibody hypervariable regions can be attached (as depicted in FIG. 2).

The term “chimeric FcR” refers to a construct composed of components of the FcR complex that have been genetically modified so that antibody hypervariable regions can be attached.

A gene sequence can be regulatable. Regulation of a gene expression can be accomplished by one of 1) alteration of gene structure: site-specific recombinases (e.g., Cre based on the Cre-loxP system) can activate gene expression by removing inserted sequences between the promoter and the gene; 2) changes in transcription either by induction (covered) or by relief of inhibition; 3) changes in mRNA stability by specific sequences incorporated in the mRNA or by siRNA; and 4) changes in translation by sequences in the mRNA. Deleted flaviviruses are also called “high-capacity” flaviviruses. These deleted flaviviruses can accommodate up to 8 kb of genetic sequences.

As used herein, the term “gene expression construct” refers to a promoter, at least a fragment of a gene of interest, and a polyadenylation signal sequence. A vector module of the present disclosure comprises a gene expression construct.

A “gene of interest” or “GOI” can be one that exerts its effect at the level of RNA or protein. Examples of genes of interest include, but are not limited to, therapeutic genes, immunomodulatory genes, virus genes, bacterial genes, protein production genes, inhibitory RNAs or proteins, and regulatory proteins. For instance, a protein encoded by a therapeutic gene can be employed in the treatment of an inherited disease, e.g., the use of a cDNA encoding the cystic fibrosis transmembrane conductance regulator in the treatment of cystic fibrosis. Moreover, the therapeutic gene can exert its effect at the level of RNA, for instance, by encoding an antisense message or ribozyme, a siRNA as is known in the art, an alternative RNA splice acceptor or donor, a protein that affects splicing or 3′ processing (e.g., polyadenylation), or a protein that affects the level of expression of another gene within the cell (i.e., where gene expression is broadly considered to include all steps from initiation of transcription through production of a processed protein), perhaps, among other things, by mediating an altered rate of mRNA accumulation, an alteration of mRNA transport, and/or a change in post-transcriptional regulation.

As used herein, the phrase “gene therapy” refers to the transfer of genetic material of interest into a host to treat or prevent a genetic or acquired disease or condition. The genetic material of interest encodes a product (e.g., a protein polypeptide, peptide, or functional RNA) whose production in vivo is desired. For example, the genetic material of interest can encode a hormone, receptor, enzyme or (poly)peptide of therapeutic value. Examples of genetic material of interest include DNA encoding: the cystic fibrosis transmembrane regulator (CFTR), Factor VIII, low density lipoprotein receptor, betagalactosidase, alpha-glactosidase, beta-glucocerebrosidase, insulin, parathyroid hormone, and alpha-1-antitrypsin.

A “gene delivery vector,” “GDV”, “gene transfer vector” or “gene transfer vehicle” means a composition including a packaged vector module of the present disclosure.

By “immune response” is preferably meant an acquired immune response, such as a cellular or humoral immune response.

In the context of the present specification, an “immunomodulatory molecule” is a polypeptide molecule that modulates, i.e., increases or decreases, a cellular and/or humoral host immune response directed to a target cell in an antigen-specific fashion, and preferably is one that decreases the host immune response. Generally, in accordance with the teachings of the present invention the immunomodulatory molecule(s) will be associated with the target cell surface membrane, e.g., inserted into the cell surface membrane or covalently or non-covalently bound thereto, after expression from the GDVs described herein. In some embodiments, the immunomodulatory molecule comprises all or afunctional portion of the CD8.alpha.-chain. For human CD8 coding sequences, see Leahy, Faseb J. 9:17-25 (1995); Leahy et al., Cell 68”1145-62 (1992); Nakayama et al., Immunogenetics 30:393-7 (1989). By “functional portion” with respect to CD8 proteins and polypeptides is meant that portion of the CD8.alpha.-chain retaining veto activity as described herein, more particularly that portion retaining the HLA-binding activity of the CD8.alpha.-chain, and specifically the immunoglobulin-like domain in the extracellular region of the CD8.alpha.-chain. Exemplary variant CD8 polypeptides are described in Gao and Jakobsen, Immunology Today 21:630-636 (2000), herein incorporated by reference. In some embodiments, the full length CD8.alpha.-chain is used. However, in some embodiments, the cytoplasmic domain is deleted. Preferably the transmembrane domain and extracellular domain are retained.

“In vivo gene therapy” and “in vitro gene therapy” are intended to encompass all past, present, and future variations and modifications of what is commonly known and referred to by those of ordinary skill in the art as “gene therapy” including ex vivo applications.

The term “introducing” or “transfection” refers to delivery of an expression vector to a host cell. A vector may be introduced into the cell by transfection, which typically means insertion of heterologous DNA or RNA into a cell by physical means (e.g., calcium phosphate transfection, electroporation, microinjection or lipofection); infection, which typically refers to introduction by way of infection agent, i.e., a virus; or transduction, which typically means stable infection of a cell with a virus or the transfer of genetic material from one microorganism to another by way of a viral agent (e.g., a bacteriophage). As set forth above, the vector may be a plasmid, virus or other vehicle.

The term “linear DNA” as used herein refers to non-circularized DNA molecules. The term “linear RNA” as used herein refers to non-circularized RNA molecules.

The term “naturally” as used herein refers to as found in nature; wild-type; innately or inherently.

The term “nucleic acid” refers to a deoxyribonucleotide or ribonucleotide polymer in either single- or double-stranded form, and unless otherwise limited, encompasses known analogues having the same essential nature of natural nucleotides in that they hybridize to single-stranded nucleic acids in a manner similar to naturally occurring nucleotides (e.g., peptide nucleic acids).

Nucleic acids are “operably linked” when placed into a functional relationship with another nucleic acid sequence. For example, a promoter or enhancer is operably linked to a coding sequence if it affects the transcription of the sequence. Generally, “operably linked” means that the DNA sequences being linked are contiguous. However, enhancers do not have to be contiguous. Linking is accomplished by ligation at convenient restriction sites. If such sites do not exist, the synthetic oligonucleotide adapters or linkers are used in accordance with conventional practice.

The term “packaging construct” or “packaging expression plasmid” refers to an engineered plasmid construct of circular, double-stranded DNA molecules, wherein the DNA molecules include at least one subset of flavivirus structural or nonstructural genes under control of a promoter. The “packaging construct” does not comprise UTRs or genetic information to enable independent virus replication to produce infections, viral particles, and/or efficient packaging of this genetic material being packages into a viral particle.

The term “pathogen” is used in a broad sense to refer to the source of any molecule that elicits an immune response. Thus, pathogens include, but are not limited to, virulent or attenuated viruses, bacteria, fungi, protozoa, parasites, cancer cells and the like. Typically, the immune response is elicited by one or more peptides produced by these pathogens. As described in detail below, genomic DNA encoding the antigenic peptides form these and other pathogens is used to generate an immune response that mimics the response to natural infection. It will also be apparent in view of the teachings herein that the methods include the use of genomic DNA obtained from more than one pathogen.

A cell that is “permissive” supports replication of a virus.

The term “plasmid” as used herein refers to an extra-chromosomal DNA molecule separate from the chromosomal DNA which is capable of replicating independently of the chromosomal DNA. In many cases, it is circular and double-stranded.

The term “polylinker” is used for a short stretch of artificially synthesized DNA which carries a number of unique restriction sites allowing the easy insertion of any promotor or DNA segment. The term “heterologous” is used for any combination of DNA sequences that is not normally found intimately associated in nature.

The term “promoter” is intended to mean a regulator region of DNA that facilitates the transcription of a particular gene. Promoters usually comprise a TATA box capable of directing RNA polymerase II to initiate RNA synthesis at the appropriate transcription initiation site for a particular coding sequence. A promoter may additionally comprise other recognition sequences generally positioned upstream or 5′ to the TATA box, referred to as upstream promoter elements, which influence the transcription initiation rate. A “constitutive promoter” refers to a promoter that allows for continual transcription of this associated gene in many cell types. An “inducible-promoter system” refers to a system that uses a regulating agent (including small molecules such as tetracycline, peptide and steroid hormones, neurotransmitters, and environmental factors such as heat, and osmolarity) to induce or to silence a gene. Such systems are “analog” in the sense that their responses are graduated, being dependent on the concentration of the regulating agent. Also, such systems are reversible with the withdrawal of the regulating agent. Activity of these promoters is induced by the presence or absence of biotic or abiotic factors. Inducible promoters are a powerful tool in genetic engineering because the expression of genes operably linked to them can be turned on or off at certain stages of development of an organism or in a particular tissue.

The term “propagate” or “propagated” as used herein refers to reproduce, multiply, or to increase in number, amount or extent by any process.

The term “purification” as used herein refers to the process of purifying or to free from foreign, extraneous, or objectionable elements.

The term “regulatory sequence” (also call “regulatory region” or “regulatory element”) as used herein refers to a promotor, enhancer or other segment of DNA where regulatory proteins such as transcription factors bind preferentially. They control gene expression and thus protein expression.

The term “restriction enzyme” (or “restriction endonuclease”) refers to an enzyme that cuts double-stranded DNA.

The term “restriction sites” or “restriction recognition sites” refers to particular sequences of nucleotides that are recognized by restriction enzymes as sites to cut the DNA molecule. The sites are generally, but not necessarily, palindromic, because restriction enzymes usually bond as homodimers) and a particular enzyme may cut between two nucleotides within its recognition site, or somewhere nearby.

The term “replication” or “replicating” as used herein refers to making an identical copy of an object such as, for example, but not limited to, a virus particle.

The term “replication deficient” as used wherein refers to the characteristic of a virus that is unable to replicate in a natural environment. A replication deficient virus is a virus that has been deleted of one or more of the genes that are essential for its replication, such as, for example, but not limited to, the E1 genes. Replication deficient viruses can be propagated in a laboratory in cell lines that express the deleted genes.

The term “target” or “targeted” as used herein refers to a biological entity, such as, for example, but not limited to, a protein, cell, organ, or nucleic acid, whose activity can be modified by an external stimulus. Depending upon the nature of the stimulus, there may be no direct change in the target, or a conformational change in the target may be induced.

As used here, a “target cell” can be present as a single entity, or can be part of a larger collection of cells. Such a “larger collection of cells” may comprise, for instance, a cell culture (either mixed or pure), a tissue (e.g., epithelial or other tissue), an organ (e.g., heart, lung, liver, gallbladder, urinary bladder, eye or other organ), an organ system (e.g., circulatory system, respiratory system, gastrointestinal system, urinary system, nervous system, integumentary system or other organ system), or an organism (e.g., a bird, mammal, particularly a human, and the like). Preferably, the organs/tissues/cells being targeted are of the circulatory system (e.g., including, but not limited to, heart, blood vessels, and blood), respiratory system (e.g., nose, pharynx, larynx, trachea, bronchi, bronchioles, lungs and the like), gastrointestinal system (e.g., including mouth, pharynx, esophagus, stomach, intestines, salivary glands, pancreas, liver, gallbladder, and others), urinary system (e.g., such as kidneys, ureters, urinary bladder, urethra, and the like), nervous system (e.g., including but not limited to, brain and spinal cord, and special sense organs, such as the eye) and integumentary system (e.g., skin). Even more preferably, the cells are selected from the group consisting of heart, blood vessels, lung, liver, gallbladder, urinary bladder, eye cells and stem cells. In an embodiment, the target cells are hepatocytes, and a method is provided for veto vector mediated transplantation of allogenic hepatocytes in a subject. In an embodiment, the target cells are keratinocytes, and a method is provided for veto vector mediated transplantation of allogeneic keratinocytes in a subject, for example, engineered skin. In an embodiment, the target cells are pancreatic islets. In an embodiment, the target cells are cardiomyocytes. In an embodiment, the target cells are kidney cells and a method is provided for veto vector mediated transplantation of allogeneic kidneys in a subject. In an embodiment, the target cells are fibroblasts, and a method is provided for veto vector mediated transplantation of allogeneic fibroblasts in a subject, for example, engineered skin. In an embodiment, the target cells are neurons. In an embodiment, the target cells are glia cells.

In particular, a target cell with which a GDV is contacted differs from another cell in that the contacted target cell comprises a particular cell-surface binding site that can be targeted by the GDV. By “particular cell-surface binding site” is meant any site (i.e., molecule or combination of molecules) present on the surface of a cell with which the GDV can interact in order to attach to the cell and, thereby, enter the cell. A particular cell-surface binding site, therefore, encompasses a cell-surface receptor and, preferably, is a protein (including a modified protein), a carbohydrate, a glycoprotein, a proteoglycan, a lipid, a mucin molecule or mucoprotein, and the like. Examples of potential cell-surface binding sites include but are not limited to: heparin and chondroitin sulfate moieties found on glycosaminoglycans; sialic acid moieties found on mucins, glycoproteins, and gangliosides; major histocompatibility complex I (MHC I) glycoproteins; common carbohydrate molecules found in membrane glucosamine, fucose, and galactose; glycoproteins, including mannose, N-acetyl-galactosamine, N-acetyl-glucosamine, fucose, and galactose; glycoproteins, such as ICAM-1, VCAM, E-selectin, P-selectin, L-selectin, and integrin molecules; and tumor-specific antigens present on cancerous cells, such as, for instance, MUC-1 tumor-specific epitopes. However, targeting a GDV to a cell is not limited to any specific mechanism of cellular interaction (i.e., interaction with a given cell-surface binding site).

The term “transfection” as used herein refers to the introduction of genetic material into a cell genetic material as DNA or RNA (for example, introduction of an isolated nucleic acid molecule or a construct of the present disclosure). The term “transduction” as used herein refers to the introduction of genetic material into a cell DNA either as DNA or by means of GDV of the present disclosure. A GDV of the present disclosure can be transduced into a target cell.

The term “vector” refers to a nucleic acid used in infection of a host cell and into which can be inserted a polynucleotide. Vectors are frequently replicons. Expression vectors permit transcription of a nucleic acid inserted therein. Some common vectors include, but are not limited to, plasmids, cosmids, viruses, phages, recombinant expression cassettes, and transposons. The term “vector” may also refer to an element which aids in the transfer of a gene from one location to another.

The term “viral DNA” or “viral RNA” as used herein refers to a sequence of DNA or RNA that is found in virus particles.

The term “viral genome” as used herein refers to the totality of the DNA or RNA that is found in virus particles, and that contains all the elements necessary for virus replication. The genome is replicated and transmitted to the virus progeny at each cycle of virus replication.

The term “virions” as used herein refers to a viral particle. Each virion consists of genetic material within a protective protein capsid.

The term “wild-type” as used herein refers to the typical form of an organism, strain, gene, protein, nucleic acid, or characteristic as it occurs in nature. Wild-type refers to the most common phenotype in the natural population. The terms “wild-type” and “naturally occurring” are used interchangeably.

FIG. 1 is a diagrammatic representation of an antibody. The heavy chains 1 and light chains 2 are indicated. VL denotes the variable region of the light chain. CL denotes the constant region of the light chain. VH denotes the variable region of the heavy chain. CH1, CH2 and CH3 denote the three different constant regions of the heavy chain. Fab denotes the arms of the antibody containing the VL, VH, CL and CH1 regions. Hinge denotes the flexible region connecting the antibody arms with constant Fc stem. Fc denotes the constant stem region that is composed of CH2 and CH3.

FIG. 2 is a diagrammatic representation of TCR and chimeric TCR constructs. (a) shows the TCR complex is composed of the variable TCR alpha- and beta-chains and the invariant CD3 molecules epsilon, gamma, delta, and zeta. (b)-(d) show different versions of chimeric TCRs in which an antibody variable region (VL and VH) is linked to a CD3 construct (TCR-1) in conjunction with CD28 components (TCR-2) and also 4-1BB or OX40 components (TCR-3).

FIG. 3 is a diagrammatic representation of various HAb designs. (a) shows bispecific antibodies are designed with deletion of the complete or partial Fc regions. (b) shows a bispecific antibody design composed of two different sets of heavy and light chains. (c) shows different versions of bispecific antibodies designs composed of two different VH/VL domains (Diabodies). (d) shows a different version of bispecific antibodies composed of two different VH/VL domains using a different linkage (Bite). (e) shows a trispecific HAb composed of three pairs of VH/VL domains (Trite). (f) shows a tetraspecific HAb is composed of four pairs of VH/VL domains (Qite). (g) shows a trispecific antibody composed of a bispecific Fab fragment, to which a third VH/VL domain is added (Tribody). (h) shows a trispecific antibody composed of a bispecific antibody to which a third VH/VL domain is added (TriAntibody). (i) shows a tetraspecific antibody composed of a bispecific Fab fragment to which two additional VH/VL domains are added (Quadbody). (j) shows a tetraspecific antibody composed of a bispecific antibody to which tow additional VH/VL domains are added (QuadAntibody).

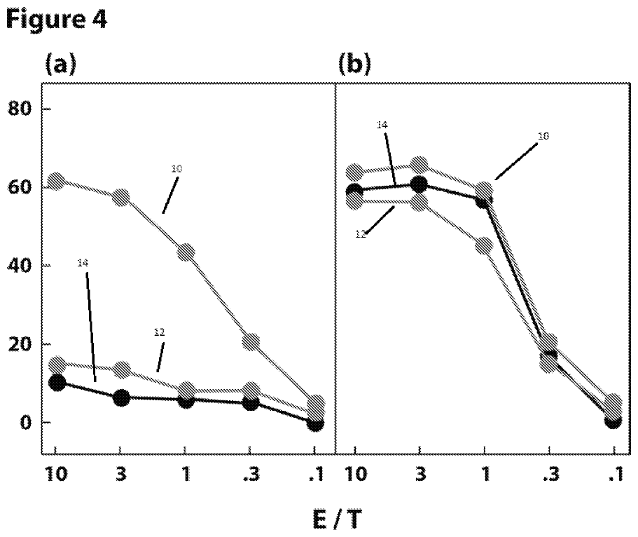

FIG. 4 is an example of targeting of an influenza infected cell to a T cell with a HAb that incorporates an anti-TCR antibody and a highly specific anti-viral antibody. Influenza virus PR/8 (10), influenza virus JAP (12) or non-infected (14) target cells were precoated with a bispecific HAb (anti-ab TCR/highly specific anti-PR/8 hemagglutinin) and tested for susceptibility to lysis by cells of a CTL line in absence of (a) or presence of (b) of the lectin PHA at different effector-target ratios (E/T).

FIG. 5 is a diagrammatic presentation of a HAb that incorporates an anti-TCR complex antibody and a broadly reactive anti-viral antibody, and a HAb that incorporates an anti-TCR complex antibody and an anti-accessory molecule antibody and a broadly reactive anti-viral antibody. (a) shows a bispecific HAb which is designed to combine an arm with anti-virus specificity and one with anti-TCR complex specificity (or anti-Fc receptor specificity). (b) shows a bispecific HAb which is designed as a Bite construct containing a VH/VL domain with an anti-virus specificity and a VH/VL domain with an anti-TCR complex specificity (or anti-Fc receptor specificity). (c) shows a trispecific HAb designed as a trite construct that combines a VH/VL domain with an anti-virus specificity, a VH/VL domain with an anti-TCR complex specificity (or anti-Fc receptor specificity) and a VH/VL domain with an anti-accessory molecule specificity, such as, but not limited to, an anti-CD28 specificity. (d) shows a trispecific HAb designed as a Tribod construct that combines an arm with an anti-virus specificity, an arm with an anti-TCR complex specificity (or anti-Fc receptor specificity) linked to a VH/VL domain with an anti-accessory molecule specificity, such as, but not limited to, an anti-CD28 specificity. (h) shows a trispecific HAb designed as a TriAntibody construct that combines an arm with an anti-virus specificity, an arm with an anti-TCR complex specificity (or anti-Fc receptor specificity) linked to a VH/VL domain with an anti-accessory molecule specificity, such as, but not limited to, an anti-CD28 specificity.

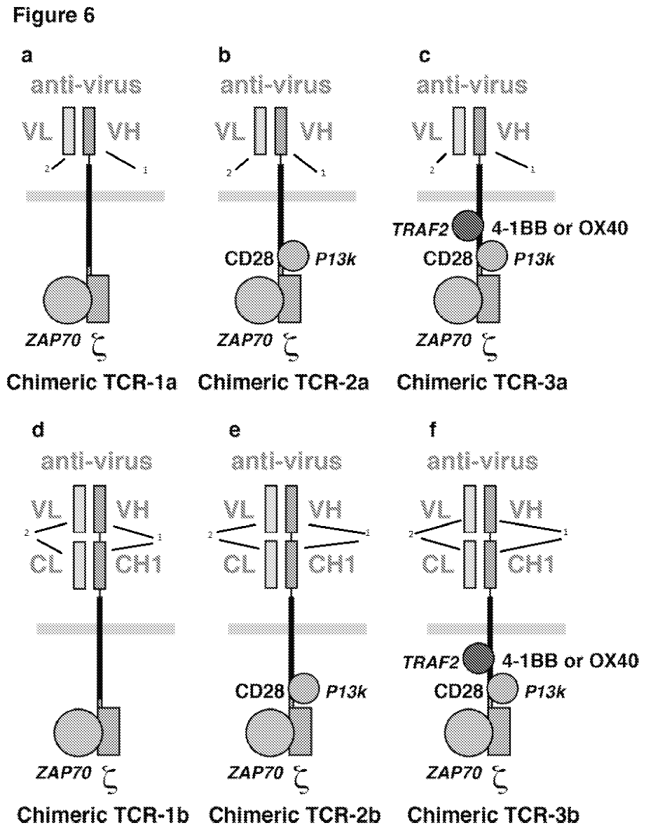

FIG. 6 is a diagrammatic presentation of a chimeric TCR that incorporates a hypervariable region of a broadly reactive anti-viral antibody. (a) show a chimeric TCR designed by linking a variable region (VL and VH) of a broadly reactive anti-viral antibody to a CD3 z-construct (TCR-1a). (b) shows a chimeric TCR designed by linking a variable region (VL and VH) of a broadly reactive anti-viral antibody to a CD3 z-construct with CD28 components (TCR-2a). (c) shows a chimeric TCR designed by linking a variable region (VL and VH) of a broadly reactive anti-viral antibody to a CD3 z-construct with CD28 components and also 41-BB or OX40 components (TCR-3a). (d) shows a chimeric TCR designed by linking a variable region (VL and VH) and a first constant region (CL and CH1) of a broadly reactive antiviral antibody to a CD3 z-construct (TCR-1b). (e) shows a chimeric TCR designed by linking a variable region (VL and VH) and a first constant region (CL and CH1) of a broadly reactive anti-viral antibody to a CD3 z-construct with CD28 components (TCR-2b). (f) shows a chimeric TCR designed by linking a variable region (VL and VH) and a first constant region (CL and CH1) of a broadly reactive anti-viral antibody to a CD3 z-construct with CD28 components and also 41-BB or OX40 components (TCR-3b).

### Hybrid Ligand Molecule

In an embodiment, the disclosure provides a hybrid ligand molecule. The hybrid ligand molecule comprises a first antibody combining site, or paratope, that binds to an effector cell receptor complex structure linked to a second antibody combining site which is a target cell-specific antibody combining site.

In an embodiment, the effector cell may be any effector cell, or combination of effector cells, as described herein. In a preferred embodiment, the effector cell is a T cell, a natural killer cell, a phagocytotic cell, and combinations thereof. In embodiment, the T cell may be an αβ T cell, a gamma delta T cell, and combinations thereof.

The effector cell receptor complex is an activating cell surface receptor. In an embodiment, the activating cell surface receptor is selected from the group consisting of a CD3 complex, FcγRI (CD64), FcγRIIA (CD32), FcγRIIB (CD32), FcγRIIIA (CD16a), FcγRIIIB (CD16b), and combinations thereof.

In an embodiment, the effector cell is a T cell and the effector cell receptor complex is a TCR complex. In a further embodiment, the effector cell receptor complex is a TCR complex and the first antibody combining site is directed to a T cell antigen receptor, or more particularly to a CD3 complex, on the surface of the T lymphocyte.

In an embodiment, the first antibody combining site is an antibody variable region which binds to a member or members of the effector cell receptor complex alone or with an accessory module.

In an embodiment, the target cell may be any target cell, or combination of target cells, as shown and described herein. In a preferred embodiment, the target cell is a virus-specific antigen. In a further embodiment, the target cell is a virus-specific antigen and the second antibody combining site binds to a protein encoded by a virus which is expressed on the surface of the target cell.

In an embodiment, viral candidates for the reactivity of the antibody variable regions of the present disclosure (and therefore target cells) are listed below. Viruses not listed here are not excluded from being targeted by the therapeutics described herein.

Viruses—Orthomyxoviruses
Influenza A—hemagglutinin (HA and neuraminidase (NA), nucleoprotein (NP1)
Mu M2, NSf, NS2 (NEP), PA, PB/, PB1-F2 and PB2
Influenza B
Influenza C

Viruses—Herpes Virus
Herpes simplex 1 (oral herpes), Herpes simplex 2 (genital herpes)—polypeptide—viral glycoproteins (designated gB, gC, gO, gE, gG, gH, gi, gJ, gK, gL, and gM) are known, and another is (gN) predicted; glycoproteins B and D

Epstein Barr (Monocucleosis, Burkitt's Lymphoma, Nasopharyngeal Carcinoma)
Epstein-Barr nuclear antigen [EBNA] 1, 2, 3A, 38, 3C, LP, and LMP; gp350/2 0, aka gp340

Cytomegalovirs
Glycoprotein B, 1 EI, pp 89, gB and pp 65 are the minimal requirements in a vaccine to induce neutralizing antibodies and cytotoxic T lymphocyte (CTL) responses. Immunization with additional proteins, e.g., for neutralizing antibodies and Elexon 4 and pp 150 for CTL responses would strengthen protective immune responses.
Varicella zoster virus (chicken pox and shingles)—recombinant proteins for gE, gI and gB genes
Kaposi's sarcoma-associated herpesvirus 8 (Kaposi's sarcoma)
Herpes 6 (A and B)
Herpes 7
Herpes B—glycoprotein B (gB)

Viruses—Papilloma Virus
For all HPV L1 capsid protein, E1, E2, E6 and E7 genes
HPV (Cervical carcinoma High-risk: 16, 18, 31, 33, 35, 39, 54, 51, 52, 56, 58, 59,
Probably high-risk: 26, 53, 66, 68, 73, 82)
HPV (common warts: 2, 7)
HPV (Plantar warts: 1, 2, 4, 63)
HPV (Flat warts: 3, 10)
HPV (Anogenital warts: 6, 11, 42, 43, 44, 55)

Viruses—Reoviridae
Rotavirus A (gastroenteritis)—VP2 and VP6 proteins

Viruses—Coronaviruses
Severe acute respiratory syndrome coronavirus (Severe Acute Respiratory Syndrome)—SARS-CoV and MERS-CoV are enveloped plus-stranded RNA viruses with a −30 kb genome encoding replicase (Rep) and the structural proteins spike (S), envelope (E), membrane (M), and nucleocapsid; MERS-CoV
Human coronavirus 229—E spike and envelope genes
Human Coronavirus NL63
Viruses—Astrovirus (gastroenteritis)—the astrovirus 870 kDa structural polyprotein
Viruses—Norovirus (gastroenteritis)—viral capsid genes, VP1 and VP2

Viruses—Fiaviviridae
Dengue fever—premembrane (prM) and envelope (E) genes
Japanese encephalitis—prM, E and NSf genes; prM and envelope (E) coding regions of JE virus
Kyasanur Forest disease
Murray Valley encephalitis
St. Louis encephalitis
Tick-borne encephalitis
West Nile encephalitis
Zika virus
Yellow Fever virus
Hepatitis C—Hepatitis C Virus Glycoprotein E2; glycoproteins E1 and E2 of hepatitis C; the core gene of HCV

Viruses—Picornaviridae—Enterovirus
Human enterovirus A (21 subtypes including some coxsackie A viruses)
Human enterovirus B (57 types including enteroviruses, coxsackie B viruses)
Human enterovirus C (14 types including some coxsackie A viruses)
Human enterovirus D (three types: EV-68, EV-70, EV-94)—VPI gene

Viruses—Picornaviridae-Rhinovirus
Human rhinovirus A (74 serotypes)
Human rhinovirus 8 (25 serotypes)
Human rhinovirus C (7 serotypes)—rhinovirus-derived VPI; the surface protein which is critically involved in infection of respiratory cells

Viruses—Picornaviridae—Hepatovirus
Hepatitis A

Viruses—Togaviridae—Alphavirus
Sindbis virus
Eastern equine encephalitis virus
Western equine encephalitis virus
Venezuelan equine encephalitis virus
Ross River virus
O'nyong'nyong virus

Viruses—Togaviridae—Rubivirus
Rubella virus

Viruses—Togaviridae—Hepevirus
Hepatitis E virus—the ORF2 protein; recombinant HEY capsid protein; the vaccine peptide has a 26 amino acids extension from the N terminal of another peptide, E2, of the HEY capsid protein

Viruses—Togaviridae—Bomaviridae
Barna disease virus—BDV nucleoprotein (BDV-N)

Viruses—Togaviridae—Filoviridae
Ebolavirus
Marburgvirus

Viruses—Togaviridae—Paramyxoviruses
Measles
Sendai virus
Human parainfluenza viruses 1 and 3
Mumps virus
Human parainfluenza viruses 2 and 4
Human respiratory syncytial virus
Newcastle disease virus
Viruses-Togaviridae-Retrovirus
HIV-gag: p18, [24, −55; pol: p31, p51, p66; env: p41, p120, p160
Hepatitis B virus
HTLVI, II

Viruses—Togaviridae—Rhabdoviruses
Rabies

Viruses—Togaviridae—Arenaviruses
Ranta virus
Korean hemorrhagic fever
Lymphocytic choriomeningitis virus
Junin
Machupo
Lassa
Sabia
Guanarito
California encephalitis
Congo—Crimean hemorrhagic fever
Rift valley fever

Viruses—Parvoviruses
Human parvovirus (B19)

In a specific embodiment, the target cells comprise one or more proteins expressed on their surface which are encoded with in a coronavirus (CoV), preferably a 3-CoV, and more preferably a 3-CoV selected from the group consisting of a SARSr virus and a MERS virus, or more preferably a SARS-1 virus, SARS-2 virus, and a MERS virus.

In an embodiment, the hybrid ligand molecules comprise a plurality of different, linked antibody combining sites, wherein at least a first of the linked antibody combining sites is configured to bind with an effector cell receptor complex and at least a second of the linked antibody combining sites is configured to bind with a target cell-specific antigen.

### Composition

In an embodiment, the disclosure provides a composition comprising a unit dose of hybrid ligand molecule. The hybrid ligand molecule may be in accordance with any embodiment or combination of embodiments of a hybrid ligand molecule as provided herein.

In an embodiment, the unit dose is dispersed in a physiologically tolerable diluent.

The composition, when contacted in an effect amount in vitro with target cells, induces destruction of the target cells by an effector cell that reacts with the target cells.

In a particular embodiment, the composition, when contacted in an effective amount in vitro with target cells in the presence of an exogenously supplied source of cytotoxic effector T lymphocytes, induces lysis of the target cells by said cytotoxic effector T lymphocytes.

### Method of Killing Infected Cells

In an embodiment, the disclosure provides a method of killing infected cells.

In an embodiment, the method comprises first providing a composition comprising a unit dose of a hybrid ligand molecule. The composition may be in accordance with any embodiment or combination of embodiments of a composition as provided herein.

The method further comprises contacting target cells, or infected cells that bear a specific antigen, with the composition in the presence of a source of effector cells whose product is activated by the first antibody combining site. The composition is present in an amount sufficient to effect binding to the effector cells and the target cells. In an embodiment, the effector cells are cytotoxic effector T lymphocytes and the target cells are tumor cells.

In an embodiment, the step of contacting requires administering the composition to a human or animal host, and preferably an infected human or animal.

The method further comprises maintaining the contact for a time period sufficient (i) for the second antibody combining site to bind to the target cells, or target cell-specific antigen, and (ii) for said first antibody combining site to bind to and activate production of the effector cells.

In an embodiment, the method further comprises periodically repeating the providing, contacting and maintaining until substantially all, or all, of the target cells have been killed.

In a particular embodiment, the effector cell is a T cell, or more specifically a phagocytotic cell.

In specific embodiments, HAbs and chimeric TCRs of the present disclosure are used to treat and/or prevent infectious diseases by recruiting immune effector cells to sites of infection, by viruses, bacteria or other infectious agents. In other embodiments, HAbs and chimeric TCRs of the disclosure are used to recruit and activate immune effector cells to act upon cells infected with viruses of certain types. In other embodiments, HAbs and chimeric TCRs of the disclosure are used to recruit and activate immune effector cells to sites of infection, by other infectious agents, such as, but not limited to, bacteria, fungal or parasites.

In one embodiment, a HAb is designed as a bispecific HAb that combines an arm with anti-virus specificity and one with anti-TCR complex specificity (FIG. 5). In one embodiment, a bispecific HAb is designed that combines an arm with anti-virus specificity and one with anti-Fc receptor specificity (FIG. 5).

In one embodiment, a HAb is designed as a Bite construct containing a VH/VL domain with an anti-virus specificity and a VH/VL domain with an anti-TCR complex specificity (FIG. 5). In one embodiment, a HAb is designed as a Bite construct containing a VH/VL domain with an anti-virus specificity and a VH/VL domain with an anti-Fc receptor specificity (FIG. 5).

In one embodiment, a trispecific HAbs is designed as a Trite construct that combines a VH/VL domain with an anti-virus specificity, a VH/VL domain with an anti-TCR complex specificity and a VH/VL domain with an anti-accessory molecule specificity, such as, but not limited to, an anti-CD28 specificity (FIG. 5). In one embodiment, a trispecific HAbs is designed as a Trite construct that combines a VH/VL domain with an anti-virus specificity, a VH/VL domain with an anti-Fc receptor specificity and a VH/VL domain with an anti-accessory molecule specificity, such as, but not limited to, an anti-CD28 specificity (FIG. 5).

In one embodiment, a trispecific HAb is designed as a Tribody construct that combines an arm with an anti-virus specificity, an arm with an anti-TCR complex specificity linked to a VH/VL domain with an anti-accessory molecule specificity, such as, but not limited to, an anti-CD28 specificity (FIG. 5). In one embodiment, a trispecific HAb is designed as a Tribody construct that combines an arm with an anti-virus specificity, an arm with an anti-Fc receptor specificity linked to VH/VL domain with an anti-accessory molecule specificity, such as, but not limited to, an anti-CD28 specificity (FIG. 5).

On one embodiment, a trispecific HAb is designed as a TriAntibody construct that combines an arm with an anti-virus specificity, an arm with an anti-TCR complex specificity linked to a VH/VL domain with an anti-accessory molecule specificity, such as, but not limited to, an anti-CD28 specificity (FIG. 5). In one embodiment, a trispecific HAb is designed as a TriAntibody construct that combines an arm with an anti-virus specificity, an arm with an anti-Fc receptor specificity linked to a VH/VL domain with an anti-accessory molecule specificity, such as, but not limited to, an anti-CD28 specificity (FIG. 5).

In one embodiment, a chimeric TCR is designed by linking a variable region (VL and VH) of a broadly reactive anti-viral antibody to a CD3 z-construct with CD28 components (FIG. 6). In one embodiment, a chimeric TCR is designed by linking a variable region (VL and VH) of a broadly reactive anti-viral antibody to a CD3 z-construct with CD28 components (FIG. 6). In one embodiment, a chimeric TCR is designed by linking a variable region (VL and VH) of a broadly reactive anti-viral antibody to a CD3 z-construct with CD8 components and also 41-BB or OX40 components (FIG. 6).

In one embodiment, a chimeric TCR is designed by linking a variable region (VL and VH) and a first constant region (CL and CH1) of a broadly reactive anti-viral antibody to a CD3 z-construct (FIG. 6). In one embodiment, a chimeric TCR is designed by linking a variable region (VL and VH) and a first constant region (CL and CH1) of broadly reactive anti-viral antibody to a CD3 z-construct with CD28 components (FIG. 6). In one embodiment, chimeric TCR is designed by linking a variable region (VL and VH) and a first constant region (CL and CH1) of a broadly reactive anti-viral antibody to a CD3 z-construct with CD28 components and also 41-BB or OX40 components (FIG. 6).

One of skill in the art will appreciate that suitable methods of administering a composition of the present disclosure to a human or animal for therapeutic purposes, e.g., gene therapy, immune therapy, vaccination, and the like (see, for example, Rosenfeld at al., Science, 252, 431 434 (1991), Jaffe et al., Clin. Res., 39(2), 302A (1991), Rosenfeld et al., Clin. Res., 39(2), 311A (1991), Berkner, BioTechniques, 6, 616 629 (1988)), are available and, although more than one route can be used to administer an antibody construct, a particular route can provide a more immediate and more effective reaction that another route. Pharmaceutically acceptable excipients are also well-known to those who are skilled in the art, and are readily available. The choice of excipient will be determined in part by the particular method used to administer antibody constructs. Accordingly, there is a wide variety of suitable formulations of an antibody construct of the present disclosure. The following formulations and methods are merely exemplary and are in no way limited. However, oral, injectable, and aerosol formulations are preferred.

Formulations suitable for oral administration can consist of (a) liquid solutions; (b) capsules, sachets or tablets; (c) suspensions in an appropriate liquid; and (d) suitable emulsions. In an embodiment, an antibody construct of the present disclosure, alone or in combination with other suitable components, can be made into aerosol formulations to be administered via inhalation. These aerosol formulations can be placed into pressurized acceptable propellants, such as dichlorodifluoromethane, propane, nitrogen, and the like. An antibody construct of the present disclosure may also be formulated as pharmaceuticals for non-pressurized preparations such as in a nebulizer or an atomizer. Formulations suitable for parenteral administration include aqueous and non-aqueous, isotonic sterile injection solutions, which can comprise anti-oxidants, buffers, bacteriostats, and solutes that render the formulation isotonic with the blood of the intended recipient, and aqueous and non-aqueous sterile suspensions that can includes suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared form sterile powders, granules, and tablets of the kind previously described.

The dose administered to an animal, particularly a human, in the context of the present disclosure will vary with the gene or other sequence of interest, the composition employed, the method of administration, and the particular site and organism being treated. The dose should be sufficient to effect a desirable response, e.g., therapeutic or immune response, within a desirable time frame.

Hence, one or more of the following routes may administer an antibody construct of the present disclosure: oral administration, injection (such as direct injection), topical, inhalation, parenteral administration, mucosal administration, intramuscular administration, intravenous administration, subcutaneous administration, intraocular administration or transdermal administration. In an embodiment, antibody constructs of the present disclosure are administered topically. In an embodiment, antibody constructs of the present disclosure are administered by inhalation. In an embodiment, antibody constructs of the present disclosure are administered by a method selected from the group consisting of parenteral, mucosal, intramuscular, intravenous, subcutaneous, intraocular, and transdermal means and combinations thereof, and are formulated for each such administration.

Typically, a physician will determine the actual dosage of antibody constructs that will be the most suitable for an individual subject and it will vary with the age, weight and response of the particular patient and severity of the condition. The specific dose level and frequency of dosage for any particular patient may be varied and will depend upon a variety of factors including the activity of the specific compound employed, the metabolic stability and length of action of the compound, the age, body weight, general health, sex, diet, mode and time of administration, rate of excretion, drug combination, the severity of the particular condition, and the host undergoing therapy.

The dose administered to an animal, particularly a human, in the context of the present disclosure, will vary with the therapeutic transgene of interest and/or the nature of the immunomodulatory molecule, the composition employed, the method of administration, and the particular site and organism being treated. However, preferably, a dose corresponding to an effective amount of an antibody construct are employed. An “effective amount” is one that is sufficient to produce the desired effect in a host, which can be monitored using several end-points known to those skilled in the art. For instance, one desired effect is nucleic acid transfer to a host cell. Such transfer can be monitored by a variety of means, including, but not limited to, a therapeutic effect (e.g., alleviation of some symptom associated with the disease, condition, disorder or syndrome being treated), or by evidence of the transferred gene or coding sequence or its expression within the host (e.g., using the polymerase chain reaction, Northern or Southern hybridizations, or transcription assays to detect the nucleic acid in host cells, or using immunoblot analysis, antibody-mediated detection, or particularized assays to detect protein or polypeptide encoded by the transferred nucleic acid, or impact in level or function due to such transfer). These methods described are by no means all-inclusive, and further methods to suit the specific application will be apparent to the ordinary skilled artisan. In this regard, it should be noted that the response of a host to the introduction of an antibody construct can vary depending on the dose of virus administered, the site of delivery, and the genetic makeup of the antibody construct as well as the transgene and the means of inhibiting an immune response.

In an embodiment, the engineered TCR and antibody are used to protect an individual against CoVs, such as but not limited to, a SARS-CoV or a MERS-CoV. In an embodiment, the virus is a SARS-CoV-1 or a SARS-CoV-2 virus. In an embodiment, the virus is a MERS-CoV virus. In an embodiment, the antibody hypervariable regions of the present disclosure are selected from one directed against CoV encoded proteins, such as but not limited to the Spike (S), Membrane (M), Nucleocaspid (N) and the Envelope (E) proteins.

### EXAMPLES

### Example 1 (FIG. 4)

An example is given that demonstrates that HAb antibodies can indeed by used to target T lymphocytes to virus-infected cells. Here, the highly specific anti-influenza monoclonal antibody 2A7 recognizes the highly variable head region of the PR/8 influenza virus, but not the highly variable head region of the JAP influenza virus. The F23.1 monoclonal antibody recognizes the beta-chain of the TCR. A bispecific HAb linking the hypervariable regions of 2A7 and F23.1 was produced. SL2 mouse cells were infected with the influenza virus PR/8 or with the influenza virus JAP. Control SL2 mouse cells were non-infected. The different cell populations were coated with the HAb and washed. They were then assayed for their susceptibility to lysis of OE4 CTLS. Figure demonstrates that the HAb can medicate lysis of the PR/9 infected cells. Yet, as the anti-virus antibody used in these studies was highly specific for the PR/8 infected cells, control cells or cells infected with the JAP influenza virus were not lysed. Yet, when the non-specific lectin was used to coat the different cells, all were lysed demonstrating that all target cells were susceptible to lysis by the OE4 CTLs. A broadly reactive anti-viral antibody would have bound to cells infected with different strains of influenza virus and would thus make them susceptible to activity of the effector T cells when incorporated into HAbs of similar design.

### Example 2 (FIG. 5)

A bispecific HAb is designed as a Bite construct. For this purpose, the variable domain of a broadly reactive anti-influenza hemagglutinin antibody, such as the variable domain (VH/VL) of the monoclonal antibody CT149, together with the variable domain (VH/VL) of an anti-human-TCR complex monoclonal antibody, such as the variable domain (VH/VL) of the monoclonal antibody OKT3 (FIG. 5b). The different fragments are cloned into an expression vector in the following order. Promotor-signal/leader peptide-VL (CT149)-spacer peptide-VH (CT149)-spacer peptide-VL (OKT3)-spacer peptide-VH (OKT3)-spacer peptide-polyadenylation site. Other antibody variable regions with similar specificities are envisioned also in their humanized form in the same order or in other orders so that the VI and VH regions of the different hypervariable regions reassemble into their proper functional configuration.

Once the expression vector, such as a eukaryotic expression vector has been assembled as described above using the necessary eukaryotic promoter and polyadenylation sites, it is transferred or transfected into a eukaryotic cell, in which the HAb is produced and from which it sis purified.

Alternative a prokaryotic expression vector is used with a prokaryotic promoter and polyadenylation site, it is transferred or transformed into bacteria, in which the HAb is produced and from which it is purified. It is also possible to produce such HAbs in plant cells using the respective expression control elements.

The HAb is being used to coat human T cells in vitro or in vivo so that they can act upon infected cells.

### Example 3 (FIG. 5)

A bispecific HAb is designed as a Bite construct. For this purpose, the variable domain of a broadly reactive anti-influenza hemagglutinin antibody, such as the variable domain (VH/VL) of the monoclonal antibody CT149, together with the variable domain (VH/VL) of an anti-human-Fc receptor complex, such as an anti-FcγRI (CD64) monoclonal antibody, such as the variable domain (VH/VL) of the monoclonal antibody 10.1 (FIG. 5b). The different fragments are cloned into an expression vector in the following order: promoter-signal/leader peptide-VL (CT149)-spacer peptide-VH (CT149)-spacer peptide-VL (10.1)-spacer peptide-VH (10.1)-spacer peptide-polyadenylation site. Other antibody variable regions with similar specificities are envisioned also in their humanized form in the same order in other orders so that the VL and VH regions of the different hypervariable regions reassemble in the proper functional configuration.

Once the expression vector, such as a eukaryotic expression vector has been assembled as described above using the necessary eukaryotic promoter and polyadenylation sites, it is transferred to transfected into a eukaryotic cell, in which the HAb is produced and form which it is purified.

Alternatively a prokaryotic expression vector is used with a prokaryotic promoter and polyadenylation site it is transferred or transformed into bacteria, in which the HAb is produced and from which it is purified. It is also possible to produce such HAbs in plant cells using the respective expression control elements.

The HAb is being used to coat Fc receptor-bearing cells in vitro or in vivo so that they can act upon infected cells.

### Example 4 (FIG. 5)

A trispecific HAb is designed as a Trite construct. For this purpose, the variable domain of a broadly reactive anti-influenza hemagglutinin antibody, such as the variable domain (VH/VL) of the monoclonal antibody CT149, together with the variable domain (VH/VL) of an anti-human-TCR complex monoclonal antibody, such as the variable domain (VH/VL) of the monoclonal antibody OKT3 (FIG. 5c).

The different fragments are cloned into an expression vector in the following order: promoter-signal/leader peptide-VL (CT149)-spacer peptide-VH (CT149)-spacer peptide-VL (OKT3)-spacer peptide-VH (OKT3)-VL (CD28.2)-spacer peptide (VH (CD28.2))-spacer peptide-polyadenylation site. Other antibody variable regions with similar specificities are envisioned also in humanized form in the same order or in other orders so that the VL and VH regions of the different hypervariable regions reassemble in the proper functional configuration.

Once the expression vector, such as a eukaryotic expression vector has been assembled as described above using the necessary eukaryotic promoter and polyadenylation sites, it is transferred or transfected into a eukaryotic cell, in which the HAb is produced and from which it is purified.

Alternatively a prokaryotic expression vector is used with a prokaryotic promoter and polyadenylation site, it is transferred or transformed into bacteria, in which the HAb is produced and from which it is purified. It is also possible to produce such HAbs in plant cells using the respective expression control elements.

### Example 5 (FIG. 5)

A bispecific HAb is designed as a hybrid antibody construct. For this purpose, the light and heavy chains of a broadly reactive anti-influenza hemagglutinin antibody, such as of CT149, are co-expressed with the light and heavy chains of an anti-TCR complex antibi, such as OKT3 in a single eukaryotic, prokaryotic or plant production cell. It may be necessary to modify the respective Ig Fc regions so that they can form dimers and/or that the different Fc can preferentially join. The antibodies used in the bispecific HAb may have been humanized prior to the production of the HAb.

### Example 6 (FIG. 6)

A chimeric TCR is designed by engineering an expression construct that links a hypervariable region of the broadly reactive anti-viral antibody, as the VL/VH fragments of CT149, to the fragments of CD3, CD28, and 4-1BB via a peptide spacer.

The different fragments are cloned into an expression vector in the following order: promoter-signal/leader peptide-VL (CT149)-spacer peptide-VH (CT149)-spacer peptide-4-1BB fragment-spacer peptide-CD28 fragment-CD3 z-polyadenylation site (FIG. 6c).

Other antibody variable regions with similar specificities are envisioned also in their humanized form in the same order or in other orders so that the VL and VH regions of the different hypervariable regions reassemble in the proper functional configuration.

The expression construct transferred into T lymphocytes so that the chimeric TCR is expressed. The expression construct is delivered by a vector, such as, but not limited to, lentivirus vector that is able to transduce T lymphocytes.

The T cell transduction with the expression vector will be performed in vitro or in vivo. In vitro transduced T cells will be either autologous harvested from a patients or heterologous T cells. Upon transduction they will be transferred into the patient for therapy against the viral infection.

While multiple embodiments of a hybrid ligand, composition and method of killing infected cells have been described in detail herein, it should be apparent that modifications and variations thereto are possible, all of which fall within the true spirit and scope of the invention. Further, since numerous modifications and changes will readily occur to those skilled in the art, it is not desired to limit the invention to the exact construction and operation shown and described, and accordingly, all suitable modifications and equivalents may be resorted to, falling within the scope of this disclosure.

What is claimed is:

1. A hybrid ligand molecule comprising a first antibody combining site that binds to an effector cell receptor complex structure of an effector cell linked to a second antibody combining site which is a target cell-specific antibody combining site.

2. The hybrid ligand molecule of claim 1, wherein the effector cell is a T lymphocyte and the effector cell receptor complex structure is a T cell receptor complex structure.

3. The hybrid ligand molecule of claim 2, wherein the first antibody combining site is directed to a T cell antigen receptor on a surface of the T lymphocyte.

4. The hybrid ligand molecule of claim 2, wherein the first antibody combining site is directed to a CD3 complex on a surface of the T lymphocyte.

5. The hybrid ligand molecule of claim 1, wherein the second antibody combining site binds to a protein encoded by a virus which is expressed on a surface of the target cell.

6. The hybrid ligand molecule of claim 5, wherein the second antibody combining site is directed to a viral protein encoded within a coronavirus and expressed on the surface of the target cell.

7. The hybrid ligand molecule of claim 1, wherein the first antibody combining site binds to a T cell receptor complex structure and is capable of activating T lymphocytes.

8. A hybrid ligand molecule comprising a first antibody combining site that binds to an activating cell surface receptor on an effector cell.

9. The hybrid ligand molecule of claim 8, wherein the effector cell is selected from the group consisting of T cells, natural killer cells, phagocytotic cells, and combinations thereof.

10. The hybrid ligand molecule of claim 9, wherein the effector cell is selected from the group consisting of alpha beta T cells, gamma delta T cells, natural killer cells, and phagocytotic cells.

11. The hybrid ligand molecule of claim 8, wherein the activating cell surface receptor is selected from the group consisting of a CD3 complex, FcγRI (CD64), FcγRIIA (CD32), FcγRIIB (CD32), FcγRIIIA (CD16a), FcγRIIIB (CD16b), and combinations thereof.

12. The hybrid ligand molecule of claim 8, wherein the activating cell surface receptor is selected from the group consisting of FcγRI (CD64), FcγRIIA (CD32), FcγRIIB (CD32), FcγRIIIA (CD16a), FcγRIIIB (CD16b), and combinations thereof.

13. A hybrid ligand molecule comprising a plurality of different, linked antibody combining sites, wherein at least a first of the plurality binds to a T cell receptor complex structure and at least a second of the plurality binds to a target cell-specific antigen.

14. A composition including hybrid ligand molecules dispersed in a physiologically tolerable diluent, said hybrid ligand molecules comprising a plurality of different, linked antibody combining sites, wherein at least a first of the plurality binds to a T cell receptor complex structure and at least a second of the plurality binds to a target cell-specific antigen,
wherein, when contacted in an effective amount in vitro with target cells in the presence of an exogenously supplied source of cytotoxic effector T lymphocytes, the fluid induces lysis of the target cells by said cytotoxic effector T lymphocytes.

15. A method of killing infected cells comprising:
(a) providing a composition containing a unit dose of hybrid ligand molecules dispersed in a physiologically tolerable diluent, the hybrid ligand molecules comprising a first antibody combining site linked to a second antibody combining site, wherein the first antibody combining site binds to an effector cell receptor complex structure and the second antibody combining site binds to a virus specific antigen, and wherein the composition induces destruction of the virus infected cells by an effector cell that reacts with cells that bear the virus specific antigen;
(b) contacting infected cells that bear the virus specific antigen with the composition in the presence of a source of effector cells whose production is activated by the first antibody combining site, wherein the composition is present in an amount sufficient to effect binding to the cytotoxic effector T lymphocytes and to the infected cells; and
(c) maintaining the contacting for a time period sufficient (i) for the second antibody combining site to bind to the virus specific antigen and (ii) for the first antibody combining site to bind to and activate production of effector cells, wherein the produced effector cells react with the cells bearing the specific antigen.

16. The method of claim 15, wherein the infected cells are tumor cells.

17. The method of claim 15, wherein the effector cells are T cells.

18. The method of claim 17, wherein the T cells are phagocytotic cells.

19. The method of claim 15, further including the step of periodically repeating steps (a)-(c) until substantially all of the infected cells have been killed.
